SEQUENCE LISTING (14 sequences)

SEQ ID NO: 1 - DNA, 1346 bp, Homo sapiens
gggtactgccgtcgccgccgcccaggccggggaggggtgcgttagtgtcaggaagcgggctgcgccgaggtcgtagcggaaccagctggcgaccccgcagaatgaaccacaagagcaagaagcgcatccgcgaggccaagcggagtgcgcggccggagctcaaggactcgctggattggacccggcacaactactacgagagcttctcgctgagcccggcggccgtggcggataacgtggaaagggcagatgctttacagctgtctgtggaagaatttgtggagcggtatgaaagaccttacaagcccgtggttttgttgaatgcgcaagagggctggtctgcgcaggagaaatggactctggagcgcctaaaaaggaaatatcggaaccagaagttcaagtgtggtgaggataacgatggctactcagtgaagatgaagatgaaatactacatcgagtacatggagagcactcgagatgatagtcccctttacatctttgacagcagctatggtgaacaccctaaaagaaggaaacttttggaagactacaaggtgccaaagtttttcactgatgaccttttccagtatgctggggagaagcgcaggcccccttacaggtggtttgtgatggggccaccacgctccggaactgggattcacatcgaccctctgggaaccagtgcctggaatgccttagttcagggccacaagcgctggtgcctgtttcctaccagcactcccagggaactcatcaaagtgacccgagacgaaggagggaaccagcaagacgaagctattacctggtttaatgttatttatccccggacacagcttccaacctggccacctgaattcaaacccctggaaatcttacaaaaaccaggagagactgtctttgtaccaggaggctggtggcatgttgtcctcaatctcgacactactatcgccatcacccaaaattttgccagcagcaccaacttccctgtggtatggcacaagacggtaagagggagaccaaagttatcaaggaaatggtataggattttgaagcaagagcaccccgagttggcagtcctcgcagactcggttgaccttcaggagtccacagggatagcttccgacagctccagcgactcttccagctcctccagctccagttcgtcagactccgactcagagtgcgagtctggatccgagggcgatgggacagtgcaccgcaggaagaagaggaggacgtgcagcatggtgggaaacggggacaccacctcccaggacgactgtgtcagcaaagagcgcagctcctccaggattagggacacttgtggaggccgggctcacccctga

SEQ ID NO: 2 - protein, 414 aa, Homo sapiens
MNHKSKKRIREAKRSARPELKDSLDWTRHNYYESFSLSPAAVADNVERADALQLSVEEFVERYERPYKPVVLLNAQEGWSAQEKWTLERLKRKYRNQKFKCGEDNDGYSVKMKMKYYIEYMESTRDDSPLYIFDSSYGEHPKRRKLLEDYKVPKFFTDDLFQYAGEKRRPPYRWFVMGPPRSGTGIHIDPLGTSAWNALVQGHKRWCLFPTSTPRELIKVTRDEGGNQQDEAITWFNVIYPRTQLPTWPPEFKPLEILQKPGETVFVPGGWWHVVLNLDTTIAITQNFASSTNFPVVWHKTVRGRPKLSRKWYRILKQEHPELAVLADSVDLQESTGIASDSSSDSSSSSSSSSSDSDSECESGSEGDGTVHRRKKRRTCSMVGNGDTTSQDDCVSKERSSSRIRDTCGGRAHP

SEQ ID NO: 3 - DNA, 1220 bp, Homo sapiens
gggtactgccgtcgccgccgcccaggccggggaggggtgcgttagtgtcaggaagcgggctgcgccgaggtcgtagcggaaccagctggcgaccccgcagaatgaaccacaagagcaagaagcgcatccgcgaggccaagcggagtgcgcggccggagctcaaggactcgctggattggacccggcacaactactacgagagcttctcgctgagcccggcggccgtggcggataacgtggaaagggcagatgctttacagctgtctgtggaagaatttgtggagcggtatgaaagaccttacaagcccgtggttttgttgaatgcgcaagagggctggtctgcgcaggagaaatggactctggagcgcctaaaaaggaaatatcggaaccagaagttcaagtgtggtgaggataacgatggctactcagtgaagatgaagatgaaatactacatcgagtacatggagagcactcgagatgatagtcccctttacatctttgacagcagctatggtgaacaccctaaaagaaggaaacttttggaagactacaaggtgccaaagtttttcactgatgaccttttccagtatgctggggagaagcgcaggcccccttacaggtggtttgtgatggggccaccacgctccggaactgggattcacatcgaccctctgggaaccagtgcctggaatgccttagttcagggccacaagcgctggtgcctgtttcctaccagcactcccagggaactcatcaaagtgacccgagacgaaggagggaaccagcaagacgaagctattacctggtttaatgttatttatccccggacacagcttccaacctggccacctgaattcaaacccctggaaatcttacaaaaaccaggagagactgtctttgtaccaggaggctggtggcatgttgtcctcaatctcgacactactatcgccatcacccaaaattttgccagcagcaccaacttccctgtggtatggcacaagacggtaagagggagaccaaagttatcaaggaaatggtataggattttgaagcaagagcaccccgagttggcagtcctcgcagactcggttgaccttcaggagtccacagggatagcttccgacagctccagcgactcttccagctcctccagctccagttcgtcagactccgactcagagaggattagggacacttgtggaggccgggctcacccctga

SEQ ID NO: 4 - protein, 372 aa, Homo sapiens
MNHKSKKRIREAKRSARPELKDSLDWTRHNYYESFSLSPAAVADNVERADALQLSVEEFVERYERPYKPVVLLNAQEGWSAQEKWTLERLKRKYRNQKFKCGEDNDGYSVKMKMKYYIEYMESTRDDSPLYIFDSSYGEHPKRRKLLEDYKVPKFFTDDLFQYAGEKRRPPYRWFVMGPPRSGTGIHIDPLGTSAWNALVQGHKRWCLFPTSTPRELIKVTRDEGGNQQDEAITWFNVIYPRTQLPTWPPEFKPLEILQKPGETVFVPGGWWHVVLNLDTTIAITQNFASSTNFPVVWHKTVRGRPKLSRKWYRILKQEHPELAVLADSVDLQESTGIASDSSSDSSSSSSSSSSDSDSERIRDTCGGRAHP

SEQ ID NO: 5 - DNA, 1589 bp, Homo sapiens
gggtactgccgtcgccgccgcccaggccggggaggggtgcgttagtgtcaggaagcgggctgcgccgaggtcgtagcggaaccagctggcgaccccgcagaatgaaccacaagagcaagaagcgcatccgcgaggccaagcggagtgcgcggccggagctcaaggactcgctggattggacccggcacaactactacgagagcttctcgctgagcccggcggccgtggcggataacgtggaaagggcagatgctttacagctgtctgtggaagaatttgtggagcggtatgaaagaccttacaagcccgtggttttgttgaatgcgcaagagggctggtctgcgcaggagaaatggactctggagcgcctaaaaaggaaatatcggaaccagaagttcaagtgtggtgaggataacgatggctactcagtgaagatgaagatgaaatactacatcgagtacatggagagcactcgagatgatagtcccctttacatctttgacagcagctatggtgaacaccctaaaagaaggaaacttttggaagactacaaggtgccaaagtttttcactgatgaccttttccagtatgctggggagaagcgcaggcccccttacaggtggtttgtgatggggccaccacgctccggaactgggattcacatcgaccctctgggaaccagtgcctggaatgccttagttcagggccacaagcgctggtgcctgtttcctaccagcactcccagggaactcatcaaagtgacccgagacgaaggagggaaccagcaagacgaagctattacctggtttaatgttatttatccccggacacagcttccaacctggccacctgaattcaaacccctggaaatcttacaaaaaccaggagagactgtctttgtaccaggaggctggtggcatgttgtcctcaatctcgacactactatcgccatcacccaaaattttgccagcagcaccaacttccctgtggtatggcacaagacggtaagagggagaccaaagttatcaaggaaatggtataggattttgaagcaagagcaccccgagttggcagtcctcgcagactcggttgaccttcaggagtccacagggatagcttccgacagctccagcgactcttccagctcctccagctccagttcgtcagactccgactcagagtgcgagtctggatccgagggcgatgggacagtgcaccgcaggaagaagaggaggacgtgcagcatggtgggaaacggggacaccacctcccaggacgactgtgtcagcaaagagcgcagctcctccaggtgacccagcaaggctgttgtctgtatggaaggacacgctcgcggcaagggcagggcctggggagggtggcctgtccagtcctgcagacaaggggaggcctgacagagcccaagaatgaggacaccctcggcacgggaacccattcacttagcgtttgctccagtagctttccctctgctaccaatgcagataaacgcggcttgttttactcaggcaagagaatgtgaatagtgccaagaaaatcctttacattatttaataaaaattgaatccattttc

SEQ ID NO: 6 - protein, 403 aa, Homo sapiens
MNHKSKKRIREAKRSARPELKDSLDWTRHNYYESFSLSPAAVADNVERADALQLSVEEFVERYERPYKPVVLLNAQEGWSAQEKWTLERLKRKYRNQKFKCGEDNDGYSVKMKMKYYIEYMESTRDDSPLYIFDSSYGEHPKRRKLLEDYKVPKFFTDDLFQYAGEKRRPPYRWFVMGPPRSGTGIHIDPLGTSAWNALVQGHKRWCLFPTSTPRELIKVTRDEGGNQQDEAITWFNVIYPRTQLPTWPPEFKPLEILQKPGETVFVPGGWWHVVLNLDTTIAITQNFASSTNFPVVWHKTVRGRPKLSRKWYRILKQEHPELAVLADSVDLQESTGIASDSSSDSSSSSSSSSSDSDSECESGSEGDGTVHRRKKRRTCSMVGNGDTTSQDDCVSKERSSSR

SEQ ID NO: 7 - DNA, 1667 bp, Mus musculus
ggcgggtctggcaggtcgctgaggccaatggcggaccgcatgaggcggtggggcggggaggaaggccgctgttcgcccaacccaaagctccggtggcggctgaggagtgaatcttctggaaggcgactttaaaggcgccgggccggagcgaagggcgtctcggggccgcgccagccagggagggagggcgcgtttggtggcaggaaacgcgagccgcgtcggggtcggggcggaaccggacggcgagcggctcacgatgaaccacaagagcaagaagcgcatccgcgaggccaagcgaagtgcgcggccggagctcaaggactcgctcgactggacccggcacaactactacgagagctacccgctgaaccccgcggccgtgccggataacgtggagagagctgatgccttacagctgtcggtgaaagagttcgtggagcgctacgagaggccttacaagcccgtggttctgctcaatgcacaagagggctggtccgcacaggagaaatggactctggagcgcctcaaaaggaaataccggaaccagaagttcaagtgcggcgaggataatgacggctactcggttaagatgaagatgaagtactacatcgagtacatggagagcacccgcgatgacagtcccctttacatcttcgatagcagctatggcgaacaccccaaaagaaggaaacttttggaagactacaaggtgcccaagtttttcacagatgatcttttccaatacgcgggggagaaacgcagacccccttacaggtggtttgtgatggggccaccgcgttctggaactgggattcacatcgaccctctggggaccagtgcctggaatgccttagttcagggtcacaagcggtggtgcctcttcccaacaaacacacccagagaactcatcaaggtgacccgagaagaaggagggaaccaacaggatgaagcaattacctggtttaatgtcatctatccccggacacagcttccaacctggccacctgaattcaaacccctggagatattacagaaaccaggagaaactgtctttgtaccagggggctggtggcatgttgtcctcaaccttgacaccaccattgccatcacccagaactttgccagcagcaccaacttccctgttgtgtggcacaagacggtaagagggagaccaaagttatcaaggaagtggtataggatcttgaaacaggagcaccctgagctggcagtcctggccgacgcagttgacctccaggagtccacaggcattgcctctgacagctccagcgactcttctagctcctctagttccagctcgtcagactcggactcagagtgtgaatctgggtcagaaggtgatgggacgacacaccgcaggaagaagaggagaacctgcagcatggtgggaaatggggacactacctcccaggatgactgtgtgagcaaagagcgcagctcctccaggtgaccacatgtggccgctgtctgtgccaggatatgctcgcagccaggcttggggggctgacacccagagaacaaggacactcagatgggagttggtagtacgtggtacagtggcttttctctgtggtcatgtcttaggggcaaaggcgtgtggatggtgccaatgaaaccctttacctatttaataaaaacggatacatttt

SEQ ID NO: 8 - protein, 403 aa, Mus musculus
MNHKSKKRIREAKRSARPELKDSLDWTRHNYYESYPLNPAAVPDNVERADALQLSVKEFVERYERPYKPVVLLNAQEGWSAQEKWTLERLKRKYRNQKFKCGEDNDGYSVKMKMKYYIEYMESTRDDSPLYIFDSSYGEHPKRRKLLEDYKVPKFFTDDLFQYAGEKRRPPYRWFVMGPPRSGTGIHIDPLGTSAWNALVQGHKRWCLFPTNTPRELIKVTREEGGNQQDEAITWFNVIYPRTQLPTWPPEFKPLEILQKPGETVFVPGGWWHVVLNLDTTIAITQNFASSTNFPVVWHKTVRGRPKLSRKWYRILKQEHPELAVLADAVDLQESTGIASDSSSDSSSSSSSSSSDSDSECESGSEGDGTTHRRKKRRTCSMVGNGDTTSQDDCVSKERSSSR

SEQ ID NO: 9 - DNA, 21 bp, Artificial Sequence
  antisense
uuaggguguucaccauagctg

SEQ ID NO: 10 - DNA, 21 bp, Artificial Sequence
  antisense
agcuuacgcugaaagcacctc

SEQ ID NO: 11 - DNA, 21 bp, Artificial Sequence
  antisence sequences
ccacaagugucccuaaucctc

SEQ ID NO: 12 - DNA, 21 bp, Artificial Sequence
  antisense
gcuauggugaacacccuaatt

SEQ ID NO: 13 - DNA, 21 bp, Artificial Sequence
  antisense
ggugcuuucagcguaagcutt

SEQ ID NO: 14 - DNA, 21 bp, Artificial Sequence
  antisense
ggauuagggacacuuguggtt

(19) United States
(12) Patent Application Publication
(10) Pub. No.: US 2012/0100144 A1
(43) Pub. Date: Apr. 26, 2012

(54) Biomarker and Treatment for Cancer

(75) Inventors: Kartik V. Desai, Singapore (SG); Edison T. Liu, Singapore (SG); Lance David Miller, Winston-Salem, NC (US)

(73) Assignee: Agency for Science, technology and Research, Connexis (SG)

(21) Appl. No.: 13/254,127

(22) PCT Filed: Mar. 2, 2010
(86) PCT No.: PCT/SG2010/000072

§ 371 Date: Aug. 31, 2011

Related U.S. Application Data
(60) Provisional application No. 61/156,819, filed on Mar. 2, 2009.

(51) Int. Cl. A61K 39/395; C07K 16/40; C07H 21/02; C40B 30/04; C12P 21/00; A61K 39/00; A61P 35/00; A61P 37/04; G01N 33/574; A61K 31/7088

(52) U.S. Cl. 424/139.1; 435/7.1; 530/387.9; 536/24.5; 514/44 A; 435/70.3; 424/185.1; 506/9

(57) ABSTRACT

A correlation between expression of JMJD6 polypeptide and breast cancer metastasis exists accordingly the present invention relates a diagnostic, prognostic and therapeutic biomarker to distinguish between early and advanced/metastatic cancer particularly breast cancer, including compounds and methods to treat the same.

### CROSS-REFERENCE TO RELATED APPLICATION

This application claims benefit of, and priority from, U.S. provisional patent application No. 61/156,819, filed on 2 Mar. 2009, the contents of which are hereby incorporated herein by reference.

### FIELD OF THE INVENTION

The present invention relates to a diagnostic, prognostic and therapeutic biomarker to distinguish between early and advanced/metastatic cancer particularly breast cancer and compounds to treat the same.

### BACKGROUND ART

Worldwide breast cancer is the second most common type of cancer and one of the most common causes of cancer death in humans. It is the most common cancer in women and makes up a third of cancer occurrence of women in the US. Common tests that provide information to assists in the diagnosis or prognosis of breast cancer include mammograms and tissue biopsy followed by combinations of histological examination, immune-histochemical detection with antibodies to estrogen receptor (ER), progesterone receptor (PR) and/or HER2/neu proteins.

Current treatment of breast cancer includes surgery, chemotherapy, radiation therapy and immunotherapy. Targeted therapy such as HER2/neu antibody first became available in the late 1990's. Other targeted therapies involve either blocking estrogen or the estrogen receptor. Estrogen is implicated in initiation and progression of breast cancer growth. Progesterone therapy is often used to block estrogen. Estrogen receptor antagonists such as tamoxifen and raloxifene have been used to treat breast cancer. Research shows that Tamoxifen becomes ineffective in 35% of patients taking the drug particularly where the breast cancer has metastasized.

Metastasis is a complex series of steps in which neoplasic cells leave the original tumor site and migrate to other parts of the body via the blood stream or the lymphatic system and start new tumors that resemble the primary tumor. Breast cancer cells are often transported through the lymphatic pathway to bone or other areas such as liver, lung or brain. It is important to determine if a cancer has metastasized because the treatment regime will vary where the cancer has metastasized. Detection of metastatic sites currently requires numerous, time consuming and costly tests.

Jumonji domain containing-6 (JMJD6) plays essential roles in embryogenesis. The protein was considered to be an important mediator in the recognition and removal of apoptotic cells. It is predominantly found in the nucleus and contains a Jumonji C (JmjC) domain. This domain is known to catalyse demethylation of histones. There remains some controversy, however, as to whether JMJD6 is a histone demethylase.

### SUMMARY OF THE INVENTION

The present invention seeks to provide novel methods of detecting and/or novel compounds for treating breast cancer metastasis to ameliorate some of the difficulties with the current detection and treatment.

We have discovered a correlation between expression of JMJD6 polypeptide and breast cancer metastasis.

Accordingly the present invention provides a method of analyzing a cell expression profile for determining whether the cell is metastatic comprising Measuring an amount of Jumonji domain containing-6 (JMJD6) nucleic acid or polypeptide in the cell; Comparing the amount of JMJD6 nucleic acid or protein present in the cell to the amount of JMJD6 nucleic acid or polypeptide in a sample isolated from normal, non-cancerous cells, wherein an amplified amount of JMJD6 nucleic acid or polypeptide in the cell relative to the amount of JMJD6 nucleic acid or polypeptide in the sample indicates advanced and/or metastatic breast cancer is present in the cell; and wherein the absence of an amplified amount of JMJD6 nucleic acid or polypeptide in the cell relative to the amount of JMJD6 nucleic acid or polypeptide in the sample indicates there is no metastatic breast cancer present in the cell.

The present invention also provides a method of detecting a metastatic state of breast cancer comprising the steps of: measuring the amount of JMJD6 nucleic acid or polypeptide in the first biological sample; and Comparing the amount of JMJD6 nucleic acid or polypeptide in the first sample with the amount of JMJD6 nucleic acid or polypeptide in a second biological sample isolated from normal, non-cancerous cells, wherein an amplified amount of JMJD6 nucleic acid or polypeptide in the first biological sample relative to the amount of JMJD6 nucleic acid or polypeptide in the second biological sample indicates breast cancer is aggressive and has metastasized and wherein the absence of an amplified amount of JMJD6 nucleic acid or polypeptide in the first biological sample relative to the amount of JMJD6 nucleic acid or polypeptide in the second biological sample indicates the breast cancer has not metastasized.

The present invention also provides an antibody capable of binding selectively a JMJD6 polypeptide set out in SEQ ID NO:2 or SEQ ID NO:4 or SEQ ID NO:6 or SEQ ID NO:8.

Another aspect of the invention provides an immunhistochemical method for measuring activity of a JMJD6 polypeptide in a test tissue section comprising: incubating the test tissue section with the antibody of the invention under conditions which allow for the formation of an antibody-antigen complex; staining the antibody-antigen complex of the test tissue section with a reagent; and analyzing the test tissue section to quantify an amount of the stained antibody-antigen complex in the test tissue section; wherein an amplified amount of the stained antibody-antigen complex relative to the amount of the stained antibody-antigen complex in a tissue section taken from normal, non-cancerous tissue indicates that the breast cancer has metastasized; and wherein the absence of an amplified amount of the stained antibody-antigen complex relative to the amount of the stained antibody-antigen complex in a tissue section taken from normal, non-cancerous tissue indicates the breast cancer has not metastasized.

The present invention also provides a method of treating breast cancer metastasis comprising administering to a patient in need of therapy an antibody of the invention.

The present invention also provides a composition comprising a therapeutically effective amount of an inhibitor of JMJD6 polynucleotide expression in cells.

The present invention also provides a kit for detecting breast cancer in cells comprising a reagent for detecting JMJD6 polynucleotide expression; a buffer and instructions for detecting whether breast cancer cells have metastasized.

The present invention further provides method for screening for antagonists of JMJD6 polynucleotide expression comprising contacting a cell expressing JMJD6 polynucleotide with a sample compound; and measuring the amount of JMJD6 polynucleotide expression in both the presence and absence of the sample compound; wherein a decrease in JMJD6 polynucleotide expression in the presence of the sample compound in relation to the JMJD6 polynucleotide expression in the absence of the sample compound indicates the sample compound is the antagonist.

The present invention further provides a method of making an antibody specific for JMJD6 polypeptide comprising isolating a JMJD6 polypeptide from a metastatic breast cancer; conjugating a JMJD6 polypeptide to a carrier protein; inducing production of an antibody of the JMJD6 polypeptide—carrier protein conjugate in a cell; and obtaining the antibody from the cell

The present invention further provides a vaccine for treating metastatic breast cancer comprising a JMJD6 polypeptide.

The present invention also provides a method of treating breast cancer metastasis comprising administering to a patient in need of therapy a vaccine of the invention.

### BRIEF DESCRIPTION OF THE DRAWINGS

FIG. 1: MCF-7, MDA MB231 and BT 549 Cells either stably expressing JMJD6 with non-disruptive scrambled RNA or Silencing RNA causing down-regulation of JMJD6 expression were assayed for four properties of cancer cells: (A) proliferation, (B) anchorage independent growth by forming colonies in soft agar, (C) motility, and (D) invasion.

FIG. 2: Isolated protein expression profiles from cells either stably expressing JMJD6 with non-disruptive scrambled RNA or Silencing RNA causing down-regulation of JMJD6 expression.

FIG. 3 Isolated protein expression profiles from cells either containing a vector over-expressing JMJD6 or Silencing RNA causing down-regulation of JMJD6 expression.

FIG. 4 Stable MCF-7 cells over-expressing JMJD6 (panel A, Isolated protein expression profiles) were injected in athymic nude mice to determine the ability of JMJD6 to form solid tumors in vivo (panel B and C). Mice harbored slow release pellets of estrogen to ensure high hormone levels that allow growth of human MCF-7 cells in mice (panel C, vector control cells).

### DETAILED DISCLOSURE

According to the invention there is provided a diagnostic, or prognostic biomarker, JMJD6, capable of distinguishing between early and advanced/metastatic breast cancer. Antagonists to expression of the JMJD6 polypeptide are able to decrease the tumorigenesis of breast cancer both in vitro and in vivo providing compounds to treat breast cancer.

Preferably the JMJD6 nucleic acid comprises nucleotide sequence SEQ ID NO:1; or SEQ ID NO:3; or SEQ ID NO: 5.

Preferably the JMJD6 polypeptide comprises nucleotide sequence SEQ ID NO:2; or SEQ ID NO:4; or SEQ ID NO: 6.

Preferably the method may further comprise bringing the nucleic acid into contact with a polynucleotide probe or primer comprising a polynucleotide sequence capable of hybridising selectively to the nucleotide sequence set out in SEQ ID No. 1 or SEQ ID NO:3, or SEQ ID NO:5 or a fragment thereof under suitable hybridising conditions; and detecting any duplex formed between the probe or primer and nucleic acid.

Preferably the method may further comprise detecting an estrogen receptor-encoding sequence wherein an amplified amount of JMJD6 nucleic acid and the estrogen receptor encoding sequence relative to the amount of JMJD6 nucleic acid and the estrogen receptor encoding sequence isolated from normal, non-cancerous cells indicates a breast cancer has metastasized; and wherein the absence of an amplified amount of both the JMJD6 nucleic acid and the estrogen receptor encoding sequence relative to the amount of JMJD6 nucleic acid and the estrogen receptor encoding sequence isolated from normal, non-cancerous cells indicates a breast cancer has not metastasized.

Preferably the method may further comprise incubating a biological sample with the antibody under conditions which allow for the formation of an antibody-antigen complex; and determining whether an antibody-antigen complex comprising the antibody is formed.

Preferably the method may further comprise using an optical microscope, obtaining an image of the stained antibody-antigen complex in the test tissue section. Further the test tissue section may comprises a cell or plurality of cells suspected to be cancerous. Further the test tissue section may be fixed.

Preferably the method may further comprise an additional second antibody capable of binding selectively a estrogen receptor polypeptide. Wherein the second antibody (capable of selectively binding the estrogen receptor polypeptide) is incubated with the biological sample or the test tissue section under conditions which allow for the formation of a second antibody-antigen complex; and determining whether the second antibody-antigen complex is formed, wherein an non-amplified amount of the second antibody-antigen complex relative to the amount of the second antibody-antigen complex in a sample taken from normal, non-cancerous tissue indicates a breast cancer has metastasized; and wherein the presence of an amplified amount of the second antibody-antigen complex relative to the amount of the second antibody-antigen complex in the sample taken from normal, non-cancerous tissue indicates a breast cancer has not metastasized. The ratio of JMJD6 to ER may be high or low, but ER is generally absent in patients with metastatic disease.

Preferably the method of treating breast cancer metastasis may further comprise administering a JMJD6 antagonist composition. Preferably the composition may be an antibody of the invention or an interfering RNA. Preferably the composition may further comprise an estrogen receptor antagonist. Preferably the estrogen receptor antagonist may comprise tamoxifen or raloxifene.

Preferably the composition may be used in treating breast cancer or for the preparation of a medicament for the treatment of breast cancer.

Preferably the reagent may be an antibody of the invention or a probe or primer comprising a polynucleotide sequence capable of hybridising selectively to the nucleotide sequence set out in SEQ ID No. 1 or SEQ ID NO:3, or SEQ ID NO:5 or a fragment thereof under suitable hybridising conditions.

In one embodiment a kit is provided for detection of the JMJD6 polypeptide. The kit may comprise an antibody of the invention or a probe or primer comprising a polynucleotide sequence capable of hybridising selectively to the nucleotide sequence set out in SEQ ID No. 1 or SEQ ID NO:3, or SEQ ID NO:5 or a fragment thereof under suitable hybridising conditions. Further, the kit may comprise an antibody capable of binding selectively to an estrogen receptor.

In one embodiment the antibody of the invention is made in a cell, Preferably the cell may comprise a host animal induced by immunisation that may include an adjuvant or a hybridoma.

In one embodiment a vaccine is provided for treatment or prophylactics of metastatic breast cancer comprising a JMJD6 polypeptide. Preferably the vaccine may further comprise at least one suitable adjuvant.

Preferably the JMJD6 polypeptide of the vaccine may comprise a sequence set out in SEQ ID No 2 or SEQ ID 4 or SEQ ID NO 6 or SEQ ID NO 8 or a homologue, variant, derivative or fragment thereof.

Preferably the vaccine may be used in treating breast cancer or for the preparation of a medicament for the treatment of breast cancer.

### JMJD6 Polynucleotides

An isolated JMJD6 nucleic acid molecule is disclosed which molecule typically encodes a JMJD6 polypeptide, allelic variant, or analog, including fragments, thereof. Specifically provided are DNA molecules selected from the group consisting of: (a) DNA molecules set out in SEQ ID NOS: 1, 3, or fragments thereof; (b) DNA molecules that hybridize to the DNA molecules defined in (a) or hybridisable fragments thereof; and (c) DNA molecules that code an expression for the amino acid sequence encoded by any of the foregoing DNA molecules.

Preferred DNA molecules according to the invention include DNA molecules comprising the sequence set out in SEQ ID NOS: 1, 3, 5, 7 or fragments thereof.

A polynucleotide is said to “encode” a polypeptide if, in its native state or when manipulated by methods well known to those skilled in the art, it can be transcribed and/or translated to produce the mRNA for and/or the polypeptide or a fragment thereof. The anti-sense strand is the complement of such a nucleic acid, and the encoding sequence can be deduced therefrom.

An “isolated” or “substantially pure” nucleic acid (e.g., an RNA, DNA or a mixed polymer) is one which is substantially separated from other cellular components which naturally accompany a native human sequence or protein, e.g., ribosomes, polymerases, many other human genome sequences and proteins. The term embraces a nucleic acid sequence or protein that has been removed from its naturally occurring environment, and includes recombinant or cloned DNA isolates and chemically synthesized analogs or analogs biologically synthesized by heterologous systems.

“JMJD6 gene sequence,” “JMJD6 gene,” “JMJD6 nucleic acids” or “JMJD6 polynucleotide” each refer to polynucleotides that are likely to be expressed in breast cancer tissue. Mutations at the JMJD6 gene sequence may be involved in metastasis of breast cancer.

The JMJD6 gene sequence is intended to include coding sequences, intervening sequences and regulatory elements controlling transcription and/or translation. The JMJD6 gene sequence is intended to include all allelic variations of the DNA sequence.

These terms, when applied to a nucleic acid, refer to a nucleic acid that encodes a JMJD6 polypeptide, fragment, homologue or variant, including, e.g., protein fusions or deletions. The nucleic acids of the present invention will possess a sequence that is either derived from, or substantially similar to a natural JMJD6 encoding gene or one having substantial homology with a natural JMJD6 encoding gene or a portion thereof. The coding sequence for human JMJD6 polypeptide is shown in SEQ ID NO: 1, 3 and 5 with the amino acid sequence shown in SEQ ID NO: 2, 4, 6 respectively. The coding sequence for murine JMJD6 polypeptide is shown in SEQ ID NO: 7, with the amino acid sequence shown in SEQ ID NO: 8.

A nucleic acid or fragment thereof is “substantially homologous” (“or substantially similar”) to another if, when optimally aligned (with appropriate nucleotide insertions or deletions) with the other nucleic acid (or its complementary strand), there is nucleotide sequence identity in at least about 60% of the nucleotide bases, usually at least about 70%, more usually at least about 80%, preferably at least about 90%, and more preferably at least about 95-98% of the nucleotide bases.

Alternatively, substantial homology or (identity) exists when a nucleic acid or fragment thereof will hybridise to another nucleic acid (or a complementary strand thereof) under selective hybridisation conditions, to a strand, or to its complement. Selectivity of hybridisation exists when hybridisation that is substantially more selective than total lack of specificity occurs. Typically, selective hybridisation will occur when there is at least about 55% identity over a stretch of at least about 14 nucleotides, preferably at least about 65%, more preferably at least about 75%, and most preferably at least about 90%. The length of homology comparison, as described, may be over longer stretches, and in certain embodiments will often be over a stretch of at least about nine nucleotides, usually at least about 20 nucleotides, more usually at least about 24 nucleotides, typically at least about 28 nucleotides, more typically at least about 32 nucleotides, and preferably at least about 36 or more nucleotides.

Thus, polynucleotides of the invention preferably have at least 75%, more preferably at least 85%, more preferably at least 90% homology to the sequences shown in the sequence listings herein. More preferably there is at least 95%, more preferably at least 98%, homology. Nucleotide homology comparisons may be conducted as described below for polypeptides. A preferred sequence comparison program is the GCG Wisconsin Bestfit program described below. The default scoring matrix has a match value of 10 for each identical nucleotide and −9 for each mismatch. The default gap creation penalty is −50 and the default gap extension penalty is −3 for each nucleotide.

In the context of the present invention, a homologous sequence is taken to include a nucleotide sequence which is at least 60, 70, 80 or 90% identical, preferably at least 95 or 98% identical at the amino acid level over at least 20, 50, 100, 200, 300, 500 or 1000 nucleotides with the nucleotides sequences set out in SEQ ID. Nos 1, 3, 5, or 7. In particular, homology should typically be considered with respect to those regions of the sequence that encode contiguous amino acid sequences known to be essential for the function of the protein rather than non-essential neighbouring sequences. Thus, for example, homology comparisons are preferrably made over regions corresponding to the jumonji demethylase domain and/or other domains of the JMJD6 amino acid sequence set out in SEQ ID NOS: 2, 4, 6 or 8 (see the section on JMJD6 polypeptides below). Preferred polypeptides of the invention comprise a contiguous sequence having greater than 50, 60 or 70% homology, more preferably greater than 80, 90, 95 or 97% homology, to one or more of the nucleotides sequences of SEQ ID NO: 1 which encodes amino acids 1 to 414 of SEQ ID NO:2, or the equivalent nucleotide sequences in SEQ ID NO:3 (that encodes amino acids 1 to 335), or SEQ ID NO:5 (that encodes amino acids 1 to 361), or SEQ ID NO:7 (that encodes amino acids 1 to 360). Preferred polynucleotides may alternatively or in addition comprise a contiguous sequence having greater than 80, 90, 95 or 97% homology to the sequence of SEQ ID NO: 1 that encodes amino acids 1 to 414 of SEQ ID NO:2 or the corresponding nucleotide sequences of SEQ ID NO:3 (that encodes amino acids 1 to 335), or SEQ ID NO:5 (that encodes amino acids 1 to 361), or SEQ ID NO:7 (that encodes amino acids 1 to 360).

Other preferred polynucleotides comprise a contiguous sequence having greater than 40, 50, 60, or 70% homology, more preferably greater than 80, 90, 95 or 97% homology to the sequence of SEQ ID NO: 1 that encodes amino acids 1 to 414 of SEQ ID No: 2 or the corresponding nucleotide sequences of SEQ ID NO:3 (that encodes amino acids 1 to 335 of SEQ ID NO: 4), or SEQ ID NO:5 (that encodes amino acids 1 to 361 of SEQ ID NO: 6), or SEQ ID NO:7 (that encodes amino acids 1 to 360 of SEQ ID NO: 8).

Nucleotide sequences are preferably at least 15 nucleotides in length, more preferably at least 20, 30, 40, 50, 100 or 200 nucleotides in length.

Generally, the shorter the length of the polynucleotide, the greater the homology required to obtain selective hybridization. Consequently, where a polynucleotide of the invention consists of less than about 30 nucleotides, it is preferred that the % identity is greater than 75%, preferably greater than 90% or 95% compared with the JMJD6 nucleotide sequences set out in the sequence listings herein. Conversely, where a polynucleotide of the invention consists of, for example, greater than 50 or 100 nucleotides, the % identity compared with the JMJD6 nucleotide sequences set out in the sequence listings herein may be lower, for example greater than 50%, preferably greater than 60 or 75%.

Nucleic acid hybridisation will be affected by such conditions as salt concentration, temperature, or organic solvents, in addition to the base composition, length of the complementary strands, and the number of nucleotide base mismatches between the hybridizing nucleic acids, as will be readily appreciated by those skilled in the art. Stringent temperature conditions will generally include temperatures in excess of 30 degrees C., typically in excess of 37 degrees C., and preferably in excess of 45 degrees C. Stringent salt conditions will ordinarily be less than 1000 mM, typically less than 500 mM, and preferably less than 200 mM. However, the combination of parameters is much more important than the measure of any single parameter. An example of stringent hybridization conditions is 65° C. and 0.1×SSC (1×SSC=0.15 M NaCl, 0.015 M sodium citrate pH 7.0).

The “polynucleotide” compositions of this invention include RNA, cDNA, genomic DNA, synthetic forms, and mixed polymers, both sense and antisense strands, and may be chemically or biochemically modified or may contain non-natural or derivatized nucleotide bases, as will be readily appreciated by those skilled in the art. Such modifications include, for example, labels, methylation, substitution of one or more of the naturally occurring nucleotides with an analog, internucleotide modifications such as uncharged linkages (e.g., methyl phosphonates, phosphotriesters, phosphoamidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule.

### JMJD6 Polypeptides

Full length JMJD6 polypeptides of the present invention have about 300 to 400 amino acids, encode a histone arginine demethylase in an animal, particularly a mammal, and include allelic variants or homologues. Full length JMJD6 polypeptides also typically comprise a jumonji domain (as defined below). JMJD6 polypeptides of the invention also include fragments and derivatives of full length JMJD6 polypeptides, particularly fragments or derivatives having substantially the same biological activity. The JMJD6 polypeptides include those comprising the amino acid sequence of SEQ ID NOS: 2, 4, 6 and 8, or allelic variants or homologues, including fragments, thereof. A particularly preferred polypeptide consists of amino acids 1 to 414 of the amino acid sequence shown as SEQ ID NO: 2 or allelic variants, homologues or fragments, thereof.

The term “polypeptide” refers to a polymer of amino acids and its equivalent and does not refer to a specific length of the product; thus, peptides, oligopeptides and proteins are included within the definition of a polypeptide. This term also does not refer to, or exclude modifications of the polypeptide, for example, glycosylations, acetylations, phosphorylations, and the like. Included within the definition are, for example, polypeptides containing one or more analogs of an amino acid (including, for example, natural amino acids, etc.), polypeptides with substituted linkages as well as other modifications known in the art, both naturally and non-naturally occurring.

In the context of the present invention, a homologous sequence is taken to include an amino acid sequence which is at least 60, 70, 80 or 90% identical, preferably at least 95 or 98% identical at the amino acid level over at least 20, 50, 100, 200, 300 or 400 amino acids with the amino acid sequences set out in SEQ ID. Nos 2, or 4, or 6, or 8. In particular, homology should typically be considered with respect to those regions of the sequence known to be essential for the function of the protein rather than non-essential neighbouring sequences. Thus, for example, homology comparisons are preferrably made over regions corresponding to the jumonji domain, site of the JMJD6 amino acid sequence set out in SEQ ID NOS: 2, or 4. The jumonji domain corresponds to approximately amino acids 1 to 361 of SEQ ID NO:2. Preferred polypeptides of the invention comprise a contiguous sequence having greater than 50, 60 or 70% homology, more preferably greater than 80 or 90% homology, to one or more of amino acids of SEQ ID NO: 2 or the corresponding regions of SEQ ID NO: 4, or SEQ ID NO:6 or SEQ ID NO:8.

Other preferred polypeptides comprise a contiguous sequence having greater than 40, 50, 60, or 70% homology, of SEQ ID No: 2 or the corresponding regions of SEQ ID NO: 4, or SEQ ID NO:6 or SEQ ID NO:8. Although homology can also be considered in terms of similarity (i.e. amino acid residues having similar chemical properties/functions), in the context of the present invention it is also possible to express homology in terms of sequence identity. The terms “substantial homology” or “substantial identity”, when referring to polypeptides, indicate that the polypeptide or protein in question exhibits at least about 70% identity with an entire naturally-occurring protein or a portion thereof, usually at least about 80% identity, and preferably at least about 90 or 95% identity.

Homology comparisons can be conducted by eye, or more usually, with the aid of readily available sequence comparison programs. These commercially available computer programs can calculate % homology between two or more sequences.

Percentage (%) homology may be calculated over contiguous sequences, i.e. one sequence is aligned with the other sequence and each amino acid in one sequence directly compared with the corresponding amino acid in the other sequence, one residue at a time. This is called an “ungapped” alignment. Typically, such ungapped alignments are performed only over a relatively short number of residues (for example less than 50 contiguous amino acids).

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Milne gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package (see below) the default gap penalty for amino acid sequences is −12 for a gap and −4 for each extension.

Calculation of maximum % homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al., 1984, Nucleic Acids Research 12:387). Examples of other software that can perform sequence comparisons include, but are not limited to, the BLAST package (see Ausubel et al., 1999 ibid—Chapter 18), FASTA (Atschul et al., 1990, J. Mol. Biol., 403-410) and the GENEWORKS suite of comparison tools. Both BLAST and FASTA are available for offline and online searching (see Ausubel et al., 1999 ibid, pages 7-58 to 7-60). However it is preferred to use the GCG Bestfit program.

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). It is preferred to use the public default values for the GCG package, or in the case of other software, the default matrix, such as BLOSUM62.

Once the software has produced an optimal alignment, it is possible to calculate % homology, preferably % sequence identity. The software typically does this as part of the sequence comparison and generates a numerical result.

JMJD6 polypeptide homologues include those having the amino acid sequences, wherein one or more of the amino acids is substituted with another amino acid which substitutions do not substantially alter the biological activity of the molecule, A JMJD6 polypeptide homologue according to the invention preferably has 80 percent or greater amino acid sequence identity to the human JMJD6 polypeptide amino acid sequence set out in SEQ ID NO: 2, 4 or 6. Examples of JMJD6 polypeptide homologues within the scope of the invention include the amino acid sequence of SEQ ID NOS: 2 wherein: (a) one or more aspartic acid residues is substituted with glutamic acid; (b) one or more isoleucine residues is substituted with leucine; (c) one or more glycine or valine residues is substituted with alanine; (d) one or more arginine residues is substituted with histidine; or (e) one or more tyrosine or phenylalanine residues is substituted with tryptophan.

Preferably “JMJD6 protein” or “JMJD6 polypeptide” refers to a protein or polypeptide encoded by the JMJD6 gene sequence, variants or fragments thereof. Also included are proteins encoded by DNA that hybridize under high or low stringency conditions, to JMJD6 encoding nucleic acids and closely related polypeptides or proteins retrieved by antisera to the JMJD6 protein(s).

“Protein modifications or fragments” are provided by the present invention for JMJD6 polypeptides or fragments thereof which are substantially homologous to primary structural sequence but which include, e.g., in vivo or in vitro chemical and biochemical modifications or which incorporate unusual amino acids. Such modifications include, for example, acetylation, carboxylation, phosphorylation, glycosylation, ubiquitination, labeling, e.g., with radionuclides, and various enzymatic modifications, as will be readily appreciated by those well skilled in the art. A variety of methods for labeling polypeptides and of substituents or labels useful for such purposes are well known in the art, and include radioactive isotopes such as ³²P, ligands which bind to labeled antiligands (e.g., antibodies), fluorophores, chemiluminescent agents, enzymes, and antiligands which can serve as specific binding pair members for a labeled ligand. The choice of label depends on the sensitivity required, ease of conjugation with the primer, stability requirements, and available instrumentation. Methods of labeling polypeptides are well known in the art. See, e.g., Sambrook et al., 1989 supra or Ausubel et al., 1992 supra.

A polypeptide “fragment,” “portion” or “segment” is a stretch of amino acid residues of at least about five to seven contiguous amino acids, often at least about seven to nine contiguous amino acids, typically at least about nine to 13 contiguous amino acids and, most preferably, at least about 20 to 30 or more contiguous amino acids.

Preferred polypeptides of the invention have substantially similar function to wild type full length JMJD6. Preferred polynucleotides of the invention encode polypeptides having substantially similar function to wild type full length JMJD6. “Substantially similar function” refers to the function of a nucleic acid or polypeptide homologue, variant, derivative or fragment of JMJD6 with reference to the wild-type JMJD6 nucleic acid or wild-type JMJD6 polypeptide.

However, non-functional forms of JMJD6 polypeptides may also be included within the scope of the invention since they may be useful, for example, as antagonists of JMJD6 function.

“Probes”. Polynucleotide polymorphisms associated with JMJD6 alleles are detected by hybridisation with a polynucleotide probe which forms a stable hybrid with that of the target sequence, under stringent to moderately stringent hybridisation and wash conditions. If it is expected that the probes will be perfectly complementary to the target sequence, stringent conditions will be used. Hybridisation stringency may be lessened if some mismatching is expected, for example, if variants are expected with the result that the probe will not be completely complementary. Conditions are chosen which rule out nonspecific/adventitious bindings, that is, which minimize noise. Since such indications identify neutral DNA polymorphisms as well as mutations, these indications need further analysis to demonstrate detection of a JMJD6 in metastatic breast cancer.

Probes for JMJD6 nucleic acid may be derived from the sequences of the JMJD6 region or its cDNAs. The probes may be of any suitable length, which span all or a portion of the JMJD6 region and which allow specific hybridisation to the jumonji domain region. If the target sequence contains a sequence identical to that of the probe, the probes may be short, e.g., in the range of about 8-30 base pairs, since the hybrid will be relatively stable under even stringent conditions. If some degree of mismatch is expected with the probe, i.e., if it is suspected that the probe will hybridize to a variant region, a longer probe may be employed which hybridises to the target sequence with the requisite specificity.

The probes will include an isolated polynucleotide attached to a label or reporter molecule and may be used to isolate other polynucleotide sequences, having sequence similarity by standard methods. For techniques for preparing and labeling probes see, e.g Sambrook et al., 1989: “Molecular Cloning: a laboratory manual. Sambrook, J., Fritsch, E. F. and Maniatis, T. (1989). Coldspring Harbour Laboratory Press, Cold spring Harbour, NY. Other similar polynucleotides may be selected by using homologous polynucleotides. Alternatively, polynucleotides encoding these or similar polypeptides may be synthesized or selected by use of the redundancy in the genetic code. Various codon substitutions may be introduced, e.g., by silent changes (thereby producing various restriction sites) or to optimize expression for a particular system. Mutations may be introduced to modify the properties of the polypeptide, perhaps to change ligand-binding affinities, interchain affinities, or the polypeptide degradation or turnover rate.

Probes comprising synthetic oligonucleotides or other polynucleotides of the present invention may be derived from naturally occurring or recombinant single- or double-stranded polynucleotides, or be chemically synthesized. Probes may also be labeled by nick translation, Klenow fill-in reaction, or other methods known in the art.

Portions of the polynucleotide sequence having at least about eight nucleotides, usually at least about 15 nucleotides, and fewer than about 6 kb, usually fewer than about 1.0 kb, from a polynucleotide sequence encoding JMJD6 are preferred as probes. The probes may also be used to determine whether mRNA encoding JMJD6 is present in a cell or tissue.

The present invention provides one or more JMJD6 polynucleotides or fragments thereof comprising mutations with respect to the wild type sequence, such as the sequence shown in SEQ ID No. 1. In a further embodiment, the present invention provides a plurality of JMJD6 polynucleotides or fragments thereof for use in screening the DNA of an individual for the presence of one or more mutations/polymorphisms. The plurality of sequences is conveniently provided immobilized to a solid substrate as is described below.

### Nucleic Acid Arrays—“DNA Chip” Technology

Polynucleotides of the invention, including probes that may be used to detect JMJD6 sequences in nucleic acid samples taken from patients, may be immobilized to a solid phase support. The probes for JMJD6 will typically form part of a library of DNA molecules that may be used to detect simultaneously a number of different genes in a given genome.

Techniques for producing immobilised libraries of DNA molecules have been described in the art. Generally, most prior art methods describe the synthesis of single-stranded nucleic acid molecule libraries, using for example masking techniques to build up various permutations of sequences at the various discrete positions on the solid substrate. U.S. Pat. No. 5,837,832, the contents of which are incorporated herein by reference, describes an improved method for producing DNA arrays immobilised to silicon substrates based on very large scale integration technology. In particular, U.S. Pat. No. 5,837,832 describes a strategy called “tiling” to synthesize specific sets of probes at spatially-defined locations on a substrate which may be used to produce the immobilised DNA libraries of the present invention. U.S. Pat. No. 5,837,832 also provides references for earlier techniques that may also be used. Thus nucleic acid probes may be synthesised in situ on the surface of the substrate.

Alternatively, single-stranded molecules may be synthesised off the solid substrate and each pre-formed sequence applied to a discrete position on the solid substrate. For example, nucleic acids may be printed directly onto the substrate using robotic devices equipped with either pins or pizo electric devices.

The library sequences are typically immobilised onto or in discrete regions of a solid substrate. The substrate may be porous to allow immobilisation within the substrate or substantially non-porous, in which case the library sequences are typically immobilised on the surface of the substrate. The solid substrate may be made of any material to which polypeptides can bind, either directly or indirectly. Examples of suitable solid substrates include flat glass, silicon wafers, mica, ceramics and organic polymers such as plastics, including polystyrene and polymethacrylate. It may also be possible to use semi-permeable membranes such as nitrocellulose or nylon membranes, which are widely available. The semi-permeable membranes may be mounted on a more robust solid surface such as glass. The surfaces may optionally be coated with a layer of metal, such as gold, platinum or other transition metal. A particular example of a suitable solid substrate is the commercially available BiaCore™ chip (Pharmacia Biosensors).

Preferably, the solid substrate is generally a material having a rigid or semi-rigid surface. In preferred embodiments, at least one surface of the substrate will be substantially flat, although in some embodiments it may be desirable to physically separate synthesis regions for different polymers with, for example, raised regions or etched trenches. It is also preferred that the solid substrate is suitable for the high density application of DNA sequences in discrete areas of typically from 50 to 100 μm, giving a density of 10000 to 40000 cm⁻².

The solid substrate is conveniently divided up into sections. This may be achieved by techniques such as photoetching, or by the application of hydrophobic inks, for example teflon-based inks (Cel-line, USA).

Discrete positions, in which each different member of the library is located may have any convenient shape, e.g., circular, rectangular, elliptical, wedge-shaped, etc.

Attachment of the nucleic acid sequences to the substrate may be by covalent or non-covalent means. The nucleic acid sequences may be attached to the substrate via a layer of molecules to which the library sequences bind. For example, the nucleic acid sequences may be labelled with biotin and the substrate coated with avidin and/or streptavidin. A convenient feature of using biotinylated nucleic acid sequences is that the efficiency of coupling to the solid substrate can be determined easily. Since the nucleic acid sequences may bind only poorly to some solid substrates, it is often necessary to provide a chemical interface between the solid substrate (such as in the case of glass) and the nucleic acid sequences. Examples of suitable chemical interfaces include hexaethylene glycol. Another example is the use of polylysine coated glass, the polylysine then being chemically modified using standard procedures to introduce an affinity ligand. Other methods for attaching molecules to the surfaces of solid substrate by the use of coupling agents are known in the art see for example WO98/49557.

Binding of complementary nucleic acid sequence to the immobilised nucleic acid library may be determined by a variety of means such as changes in the optical characteristics of the bound nucleic acid (i.e. by the use of ethidium bromide) or by the use of labelled nucleic acids, such as polypeptides labelled with fluorophores. Other detection techniques that do not require the use of labels include optical techniques such as optoacoustics, reflectometry, ellipsometry and surface plasmon resonance (SPR)—see WO97/49989, incorporated herein by reference.

Thus the present invention provides a solid substrate having immobilized thereon at least one polynucleotide of the present invention, for example JMJD6 polynucleotides. In a preferred embodiment the solid substrate further comprises polynucleotides derived from genes other than the JMJD6 gene such as a probe to the estrogen receptor polynucleotide.

Any JMJD6 nucleic acid specimen, in purified or non-purified form, can be utilised as the starting nucleic acid or acids.

PCR is one such process that may be used to amplify JMJD6 gene sequences. This technique may amplify, for example, DNA or RNA, including messenger RNA, wherein DNA or RNA may be single stranded or double stranded. In the event that RNA is to be used as a template, enzymes, and/or conditions optimal for reverse transcribing the template to DNA would be utilized. In addition, a DNA-RNA hybrid that contains one strand of each may be utilized. A mixture of nucleic acids may also be employed, or the nucleic acids produced in a previous amplification reaction described herein, using the same or different primers may be so utilised.

The specific nucleic acid sequence to be amplified, i.e., the polymorphic gene sequence, may be a fraction of a larger molecule or can be present initially as a discrete molecule, so that the specific sequence constitutes the entire nucleic acid. It is not necessary that the sequence to be amplified is present initially in a pure form; it may be a minor fraction of a complex mixture, such as contained in whole human DNA.

DNA utilized herein may be extracted from a body sample, such as blood, tissue material, breast tissue and the like by a variety of techniques such as that described by Maniatis, et. al. in Molecular Cloning: A Laboratory Manual, Cold Spring Harbor, N.Y., p 280-281, 1982). If the extracted sample has not been purified, it may be treated before amplification with an amount of a reagent effective to open the cells, or animal cell membranes of the sample, and to expose and/or separate the strand(s) of the nucleic acid(s). This lysing and nucleic acid denaturing step to expose and separate the strands will allow amplification to occur much more readily.

The deoxyribonucleotide triphosphates dATP, dCTP, dGTP and dTTP are added to the synthesis mixture, either separately or together with the primers, in adequate amounts and the resulting solution is heated to about 90 degrees-100 degrees C. from about 1 to 10 minutes, preferably from 1 to 4 minutes. After this heating period, the solution is allowed to cool, which is preferable for the primer hybridization. To the cooled mixture is added an appropriate agent for effecting the primer extension reaction (called herein “agent for polymerization”), and the reaction is allowed to occur under conditions known in the art. The agent for polymerization may also be added together with the other reagents if it is heat stable. This synthesis (or amplification) reaction may occur at room temperature up to a temperature above which the agent for polymerization no longer functions. Thus, for example, if DNA polymerase is used as the agent, the temperature is generally no greater than about 40 degree C. Most conveniently the reaction occurs at room temperature.

Specific oligonucleotide primers derived from JMJD6 gene sequence may be useful in determining whether a subject is at risk of suffering from the ailments described herein. Primers direct amplification of a target polynucleotide (eg JMJD6 or JMJD6 and estrogen receptor) prior to sequencing. Primers used in any diagnostic assays derived from the present invention should be of sufficient length and appropriate sequence to provide initiation of polyrmerisation. Environmental conditions conducive to synthesis include the presence of nucleoside triphosphates and an agent for polymerisation, such as DNA polymerase, and a suitable temperature and pH.

Primers are preferably single stranded for maximum efficiency in amplification, but may be double stranded. If double stranded, primers may be first treated to separate the strands before being used to prepare extension products. Primers should be sufficiently long to prime the synthesis of JMJD6 or JMJD6 and estrogen receptor extension products in the presence of the inducing agent for polymerization. The exact length of a primer will depend on many factors, including temperature, buffer, and nucleotide composition. Oligonucleotide primers will typically contain 12-20 or more nucleotides, although they may contain fewer nucleotides.

Primers that may be used in diagnostic assays derived from the present invention should be designed to be substantially complementary to each strand of the JMJD6 genomic gene sequence. This means that the primers must be sufficiently complementary to hybridise with their respective strands under conditions that allow the agent for polymerisation to perform. In other words, the primers should have sufficient complementarity with the 5′ and 3′ sequences flanking the detection site to hybridise therewith and permit amplification of the JMJD6 genomic gene sequence.

Oligonucleotide primers of the invention employed in the PCR amplification process that is an enzymatic chain reaction that produces exponential quantities of JMJD6 gene sequence relative to the number of reaction steps involved. Typically, one primer will be complementary to the negative (−) strand of the JMJD6 gene sequence and the other is complementary to the positive (+) strand. Annealing the primers to denatured nucleic acid followed by extension with an enzyme, such as the large fragment of DNA polymerase I (Klenow) and nucleotides, results in newly synthesised + and − strands containing the target a JMJD6 or JMJD6 and estrogen receptor gene sequence. Because these newly synthesized sequences are also templates, repeated cycles of denaturing, primer annealing, and extension results in exponential production of the region (i.e., the JMJD6 or JMJD6 and estrogen receptor gene sequence) defined by the primers. The product of the chain reaction is a discreet nucleic acid duplex with termini corresponding to the ends of the specific primers employed.

Oligonucleotide primers may be prepared using any suitable method, such as conventional phosphotriester and phosphodiester methods or automated embodiments thereof. In one such automated embodiment, diethylphosphoramidites are used as starting materials and may be synthesized as described by Beaucage, et al., Tetrahedron Letters, 22:1859-1862, 1981. One method for synthesising oligonucleotides on a modified solid support is described in U.S. Pat. No. 4,458,066.

The agent for polymerisation may be any compound or system which will function to accomplish the synthesis of primer extension products, including enzymes. Suitable enzymes for this purpose include, for example, E. coli DNA polymerase I, Klenow fragment of E. coli DNA polymerase, polymerase muteins, reverse transcriptase, other enzymes, including heat-stable enzymes (ie, those enzymes which perform primer extension after being subjected to temperatures sufficiently elevated to cause denaturation), such as Taq polymerase. Suitable enzyme will facilitate combination of the nucleotides in the proper manner to form the primer extension products that are complementary to each JMJD6 gene sequence nucleic acid strand. Generally, the synthesis will be initiated at the 3′ end of each primer and proceed in the 5′ direction along the template strand, until synthesis terminates, producing molecules of different lengths.

The newly synthesised JMJD6 strand and its complementary nucleic acid strand will form a double-stranded molecule under hybridizing conditions described above and this hybrid is used in subsequent steps of the process. In the next step, the newly synthesized double-stranded molecule (JMJD6 or estrogen receptor) is subjected to denaturing conditions using any of the procedures described above to provide single-stranded molecules.

The steps of denaturing, annealing, and extension product synthesis can be repeated as often as needed to amplify the target polymorphic gene sequence nucleic acid sequence to the extent necessary for detection. The amount of the specific nucleic acid sequence produced will accumulate in an exponential fashion. Amplification is described in PCR. A Practical Approach, ILR Press, Eds. M. J. McPherson, P. Quirke, and G. R. Taylor, 1992. This may also be achieved via real time PCR as known in the art.

The JMJD6 amplification products may be detected by Southern blot analysis, without using radioactive probes. In such a process, for example, a small sample of DNA containing a very low level of the nucleic acid sequence of the JMJD6 gene sequence is amplified, and analyzed via a Southern blotting technique or similarly, using dot blot analysis. The use of non-radioactive probes or labels is facilitated by the high level of the amplified signal. Alternatively, probes used to detect the amplified products can be directly or indirectly detectably labelled, as described herein.

Sequences amplified by the methods of the invention can be further evaluated, detected, cloned, sequenced, and the like, either in solution or after binding to a solid support, by any method usually applied to the detection of a specific DNA sequence such as PCR, oligomer restriction (Saiki, et. al., Bio/Technology, 3:1008-1012, 1985), allele-specific oligonucleotide (ASO) probe analysis (Conner, et. al., Proc. Natl. Acad. Sci. U.S.A., 80:278, 1983), oligonucleotide ligation assays (OLAs) (Landgren, et. al., Science, 241:1007, 1988), and the like. Molecular techniques for DNA analysis have been reviewed (Landgren, et. al., Science, 242:229-237, 1988).

Preferably, the method of amplifying JMJD6 is by PCR, as described herein or real time PCR and as is commonly used by those of ordinary skill in the art. Alternative methods of amplification have been described and can also be employed as long as the JMJD6 gene sequence amplified by PCR using primers of the invention is similarly amplified by the alternative means. Such alternative amplification systems include but are not limited to self-sustained sequence replication, which begins with a short sequence of RNA of interest and a T7 promoter. Reverse transcriptase copies the RNA into cDNA and degrades the RNA, followed by reverse transcriptase polymerizing a second strand of DNA. Another nucleic acid amplification technique is nucleic acid sequence-based amplification (NASBA) which uses reverse transcription and T7 RNA polymerase and incorporates two primers to target its cycling scheme. NASBA can begin with either DNA or RNA and finish with either, and amplifies to 10⁸ copies within 60 to 90 minutes. Alternatively, nucleic acid can be amplified by ligation activated transcription (LAT). LAT works from a single-stranded template with a single primer that is partially single-stranded and partially double-stranded. Amplification is initiated by ligating a cDNA to the promoter oligonucleotide and within a few hours, amplification is 10⁸ to 10⁹ fold. The QB replicase system can be utilized by attaching an RNA sequence called MDV-1 to RNA complementary to a DNA sequence of interest. Upon mixing with a sample, the hybrid RNA finds its complement among the specimen's mRNAs and binds, activating the replicase to copy the tag-along sequence of interest. Another nucleic acid amplification technique, ligase chain reaction (LCR), works by using two differently labeled halves of a sequence of interest that are covalently bonded by ligase in the presence of the contiguous sequence in a sample, forming a new target. The repair chain reaction (RCR) nucleic acid amplification technique uses two complementary and target-specific oligonucleotide probe pairs, thermostable polymerase and ligase, and DNA nucleotides to geometrically amplify targeted sequences. A 2-base gap separates the oligonucleotide probe pairs, and the RCR fills and joins the gap, mimicking normal DNA repair. Nucleic acid amplification by strand displacement activation (SDA) utilizes a short primer containing a recognition site for hincII with short overhang on the 5′ end that binds to target DNA. A DNA polymerase fills in the part of the primer opposite the overhang with sulfur-containing adenine analogs. HincII is added but only cuts the unmodified DNA strand. A DNA polymerase that lacks 5′ exonuclease activity enters at the site of the nick and begins to polymerize, displacing the initial primer strand downstream and building a new one which serves as more primer. SDA produces greater than 10⁷-fold amplification in 2 hours at 37 degrees C. Unlike PCR and LCR, SDA does not require instrumented temperature cycling. Another amplification system useful in the method of the invention is the QB Replicase System. Although PCR is the preferred method of amplification if the invention, these other methods can also be used to amplify the JMJD6 or JMJD6 and estrogen receptor gene sequence as described in the method of the invention.

A “tissue sample”, as used herein, refers to a biological sample obtained from a tissue in the body, for example a biopsy. In a preferred embodiment the tissue sample is of a tumor. Frequently the tissue sample will be a “clinical sample,” which is a sample derived from a patient such as a fine needle biopsy sample. A “tissue sample” may also include a section of tissue such as a section taken from a frozen or fixed tumor. Tissue samples can be obtained from tumors of the breast, or breast cancer tumours located at other sites for example but not limited to bladder, brain, uterus, cervix, colon, rectum, esophagus, mouth, head, skin, kidney, lung, ovary, neck, pancreas, prostate, testis, liver and stomach. The tissue sample may be present on a tissue array or may comprise a whole tissue section. An “evenly matched” tissue sample is a tissue sample of the same type (i.e. comprising the same types of cells from the same type of tumour from the same type of subject). “Evenly matched” tissue samples can be used to provide reference profiles in the methods provided herein. The evenly matched tissue can be used as a sample isolated from normal, non-cancerous cells.

A “tumor” refers to an abnormal growth of tissue that may be comprised of cells that for example, express the estrogen receptor or epidermal growth factor receptor on their cellular membranes. Tumors may be present, for example, in the breast, bladder, brain, uterus, cervix, colon, rectum, esophagus, head, skin, kidney, lung (including Non Small Cell Lung Cancer), ovary, neck, pancreas, prostate, testis, liver and stomach.

### Antibodies

The present invention also provides labelled and unlabeled monoclonal and polyclonal antibodies specific for JMJD6 polypeptides of the invention and immortal cell lines that produce a monoclonal antibody of the invention. Antibody preparation according to the invention involves: (a) conjugating a JMJD6 polypeptide to a carrier protein; (b) immunizing a host animal with the JMJD6 polypeptide fragment-carrier protein conjugate of step (a) admixed with an adjuvant; and (c) obtaining antibody from the immunized host animal.

According to the invention, JMJD6 polypeptide produced recombinantly or by chemical synthesis, and fragments or other derivatives or analogs thereof, including fusion proteins, may be used as an immunogen to generate antibodies that recognize the JMJD6 polypeptide. Such antibodies include but are not limited to polyclonal, monoclonal, chimeric, single chain, Fab fragments, and a Fab expression library.

Thus, the present invention also provides polyclonal and/or monoclonal antibodies and fragments thereof, and immunologic binding equivalents thereof, which are capable of specifically binding to the JMJD6 polypeptides and fragments thereof or to polynucleotide sequences from the jumonji domain region, particularly from the JMJD6 gene sequence or a portion thereof. Such antibodies thus include for example, but are not limited to polyclonal, monoclonal, chimeric, single chain, Fab fragments, and a Fab expression library. Production of antibodies specific for JMJD6 polypeptides or fragments thereof is described below.

A molecule is “antigenic” when it is capable of specifically interacting with an antigen recognition molecule of the immune system, such as an immunoglobulin (antibody) or T cell antigen receptor. An antigenic polypeptide contains at least about 5, and preferably at least about 10, amino acids. An antigenic portion of a molecule can be that portion that is immunodominant for antibody or T cell receptor recognition, or it can be a portion used to generate an antibody to the molecule by conjugating the antigenic portion to a carrier molecule for immunization. A molecule that is antigenic need not be itself immunogenic, i.e., capable of eliciting an immune response without a carrier.

An “antibody” is any immunoglobulin, including antibodies and fragments thereof, that binds a specific epitope. The term encompasses polyclonal, monoclonal, and chimeric antibodies, the last mentioned described in further detail in U.S. Pat. Nos. 4,816,397 and 4,816,567, as well as antigen binding portions of antibodies, including Fab, F(ab′)₂ and F(v) (including single chain antibodies). Accordingly, the phrase “antibody molecule” in its various grammatical forms as used herein contemplates both an intact immunoglobulin molecule and an immunologically active portion of an immunoglobulin molecule containing the antibody combining site. An “antibody combining site” is that structural portion of an antibody molecule comprised of heavy and light chain variable and hypervariable regions that specifically binds an antigen.

Exemplary antibody molecules are intact immunoglobulin molecules, substantially intact immunoglobulin molecules and those portions of an immunoglobulin molecule that contains the paratope, including those portions known in the art as Fab, Fab′, F(ab′)₂ and F(v), which portions are preferred for use in the therapeutic methods described herein.

Fab and F(ab′)₂ portions of antibody molecules are prepared by the proteolytic reaction of papain and pepsin, respectively, on substantially intact antibody molecules by methods that are well-known. See for example, U.S. Pat. No. 4,342,566. Fab′ antibody molecule portions are also well-known and are produced from F(ab′)₂ portions followed by reduction of the disulfide bonds linking the two heavy chain portions as with mercaptoethanol, and followed by alkylation of the resulting protein mercaptan with a reagent such as iodoacetamide. An antibody containing intact antibody molecules is preferred herein.

The phrase “monoclonal antibody” in its various grammatical forms refers to an antibody having only one species of antibody combining site capable of immunoreacting with a particular antigen. A monoclonal antibody thus typically displays a single binding affinity for any antigen with which it immunoreacts. A monoclonal antibody may therefore contain an antibody molecule having a plurality of antibody combining sites, each immunospecific for a different antigen; e.g., a bi-specific (chimeric) monoclonal antibody.

The term “adjuvant” refers to a compound or mixture that enhances the immune response to an antigen. An adjuvant can serve as a tissue depot that slowly releases the antigen and also as a lymphoid system activator that non-specifically enhances the immune response [Hood et al., in Immunology, p. 384, Second Ed., Benjamin/Cummings, Menlo Park, Calif. (1984)]. Often, a primary challenge with an antigen alone, in the absence of an adjuvant, will fail to elicit a humoral or cellular immune response. Adjuvants include, but are not limited to, complete Freund's adjuvant, incomplete Freund's adjuvant, saponin, mineral gels such as aluminum hydroxide, surface active substances such as lysolecithin, pluronic polyols, polyanions, peptides, oil or hydrocarbon emulsions, keyhole limpet hemocyanins, dinitrophenol, and potentially useful human adjuvants such as BCG (bacille Calmette-Guerin) and Corynebacterium parvum. Preferably, the adjuvant is pharmaceutically acceptable.

Various procedures known in the art may be used for the production of polyclonal antibodies to JMJD6 polypeptide, or fragment, derivative or analog thereof. For the production of antibody, various host animals can be immunized by injection with the JMJD6 polypeptide, or a derivative (e.g., fragment or fusion protein) thereof, including but not limited to rabbits, mice, rats, sheep, goats, etc. In one embodiment, the JMJD6 polypeptide or fragment thereof can be conjugated to an immunogenic carrier, e.g., bovine serum albumin (BSA) or keyhole limpet hemocyanin (KLH). Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum.

For preparation of monoclonal antibodies directed toward the JMJD6 polypeptide, or fragment, analog, or derivative thereof, any technique that provides for the production of antibody molecules by continuous cell lines in culture may be used. These include but are not limited to the hybridoma technique originally developed by Kohler et al., Nature, 256:495-497 (1975), as well as the trioma technique, the human B-cell hybridoma technique [Kozbor et al., Immunology Today, 4:72 (1983)], and the EBV-hybridoma technique to produce human monoclonal antibodies [Cole et al., in Monoclonal Antibodies and Cancer Therapy, pp. 77-96, Alan R. Liss, Inc., (1985)]. Immortal, antibody-producing cell lines can be created by techniques other than fusion, such as direct transformation of B lymphocytes with oncogenic DNA, or transfection with Epstein-Barr virus. See, e.g., M. Schreier et al., “Hybridoma Techniques” (1980); Hammerling et al., “Monoclonal Antibodies And T-cell Hybridomas” (1981); Kennett et al., “Monoclonal Antibodies” (1980); see also U.S. Pat. Nos. 4,341,761; 4,399,121; 4,427,783; 4,444,887; 4,451,570; 4,466,917; 4,472,500; 4,491,632; and 4,493,890.

In an additional embodiment of the invention, monoclonal antibodies can be produced in germ-free animals utilizing recent technology (PCT/US90/02545). According to the invention, human antibodies may be used and can be obtained by using human hybridomas [Cote et al., Proc. Natl. Acad. Sci. USA, 80:2026-2030 (1983)] or by transforming human B cells with EBV virus in vitro (Cole et al., 1985, supra). In fact, according to the invention, techniques developed for the production of “chimeric antibodies” [Morrison et al., J. Bacteriol., 159-870 (1984); Neuberger et al., Nature, 312:604-608 (1984); Takeda et al., Nature, 314:452-454 (1985)] by splicing the genes from a mouse antibody molecule specific for a JMJD6 polypeptide together with genes from a human antibody molecule of appropriate biological activity can be used; such antibodies are within the scope of this invention. Such human or humanized chimeric antibodies are preferred for use in therapy of human diseases or disorders (described infra), since the human or humanized antibodies are much less likely than xenogenic antibodies to induce an immune response, in particular an allergic response, themselves.

According to the invention, techniques described for the production of single chain antibodies (U.S. Pat. No. 4,946,778) can be adapted to produce JMJD6 polypeptide-specific single chain antibodies. An additional embodiment of the invention utilizes the techniques described for the construction of Fab expression libraries [Huse et al., Science, 246:1275-1281 (1989)] to allow rapid and easy identification of monoclonal Fab fragments with the desired specificity for a JMJD6 polypeptide, or its derivatives, or analogs.

Antibody fragments which contain the idiotype of the antibody molecule can be generated by known techniques. For example, such fragments include but are not limited to: the F(ab′)₂ fragment which can be produced by pepsin digestion of the antibody molecule; the Fab′ fragments which can be generated by reducing the disulfide bridges of the F(ab′)₂ fragment, and the Fab fragments which can be generated by treating the antibody molecule with papain and a reducing agent.

In the production of antibodies, screening for the desired antibody can be accomplished by techniques known in the art, e.g., radioimmunoassay, ELISA (enzyme-linked immunosorbent assay), “sandwich” immunoassays, immunoradiometric assays, gel diffusion precipitin reactions, immunodiffusion assays, in situ immunoassays (using colloidal gold, enzyme or radioisotope labels, for example), Western blots, precipitation reactions, agglutination assays (e.g., gel agglutination assays, hemagglutination assays), complement fixation assays, immunofluorescence assays, protein A assays, and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labelled. Many means are known in the art for detecting binding in an immunoassay and are within the scope of the present invention. For example, to select antibodies that recognize a specific epitope of a JMJD6 polypeptide, one may assay generated hybridomas for a product that binds to a JMJD6 polypeptide fragment containing such epitope.

An exemplary antibody may include an affinity-purified rabbit anti-peptide LQYENVDEDSSDSDA antibody.

The foregoing antibodies can be used in methods known in the art relating to the localization and activity of the JMJD6 polypeptide, for Western blotting, imaging JMJD6 polypeptide in situ, measuring levels thereof in appropriate physiological samples, etc.

In a specific embodiment, antibodies are developed by immunizing rabbits with synthetic peptides predicted by the protein sequence or with recombinant proteins made using bacterial expression vectors. The choice of synthetic peptides is made after careful analysis of the predicted protein structure, as described above. In particular, peptide sequences between putative cleavage sites are chosen. Synthetic peptides are conjugated to a carrier such as KLH hemocyanin or BSA using carbodiimide and used in Freunds adjuvant to immunize rabbits. In order to prepare recombinant protein, the pGEX vector can be used to express the polypeptide. Alternatively, one can use only hydrophilic domains to generate the fusion protein. The expressed protein will be prepared in quantity and used to immunize rabbits in Freunds adjuvant.

In yet another embodiment, recombinant JMJD6 polypeptide is used to immunize rabbits, and the polyclonal antibodies are immunopurified prior to further use. The purified antibodies are particularly useful for semi-quantitative assays, particularly for detecting the presence of JMJD6 polypeptide.

Preferably, the anti-modulator antibody used in the diagnostic and therapeutic methods of this invention is an affinity-purified polyclonal antibody. More preferably, the antibody is a monoclonal antibody (mAb). In addition, it is preferable for the anti-modulator antibody molecules used herein be in the form of Fab, Fab′, F(ab′)₂ or F(v) portions of whole antibody molecules.

In a preferred embodiment of the invention, antibodies will immunoprecipitate JMJD6 proteins from solution as well as react with JMJD6 protein on Western or immunoblots of polyacrylamide gels. In another preferred embodiment, antibodies will detect JMJD6 proteins in paraffin or frozen tissue sections, using immunocytochemical techniques.

Preferred embodiments relating to methods for detecting JMJD6 or its mutations include enzyme linked immunosorbent assays (ELISA), radioimmunoassays (RIA), immunoradiometric assays (IRMA) and immunoenzymatic assays (IEMA), including sandwich assays using monoclonal and/or polyclonal antibodies.

### Immunohistochemistry

Various automated sample processing, scanning and analysis systems suitable for use with immunohistochemistry are known in the art. Such systems may include automated staining and microscopic scanning, computerized image analysis, serial section comparison (to control for variation in the orientation and size of a sample), digital report generation, and archiving and tracking of samples (such as slides on which tissue sections are placed). Cellular imaging systems are commercially available that combine conventional light, fluorescent or confocal microscopes with digital image processing systems to perform quantitative analysis on cells and tissues, including immunostained samples. See, e.g., the CAS-200 system (Becton, Dickinson & Co.); BLISS and IHCscore of Bacus Laboratories, Inc. (Lombard, 1H); ACIS of Clarient, Inc. (San Juan Capistrano, Calif.); iVision and GenoMx of BioGenex (San Ramon, Calif.); ScanScope of Aperio Technologies (Vista, Californis), and LSC Laser Scanning Cytometer of CompuCyte Corporation (Cambridge, Mass.).

Tissue Preparation Tissue samples are obtained from the body and include cells and extracellular matter. Tissue samples may be from humans or non human animals. Tissue samples can be from any organ and may include disease states of such organs. Tissue samples such as tumor biopsies can be obtained using known procedures, such as a needle biopsy (See Kim, C. H. et al. J. Virol. 66:3879-3882 (1992)); Biswas, B. et al. Annals NY Acad. Sci. 590:582-583 (1990)); Biswas, B. et al. J. Clin. Microbial. 29:2228-2233 (1991). The tissue is to be processed in a manner that allows accurate detection and quantitation of JMJD6 protein. The tissue sample may be prepared in a tissue microarray format and sectioned or may comprise a whole tissue section. Sections are typically prepared on microscope slides. For example, paraffin-embedded formalin-fixed specimens may be prepared, cores taken from separate areas of the specimens, each core arrayed into a recipient block, and sections cut and processed as previously described, for example, in Konenen, J. et al., Tissue microarrays for high-throughput molecular profiling of tumor specimens, (1987) Nat. Med. 4:844-7. When analyzing tissue samples from individuals, it may be important to prevent any changes, physiological processing or degredation, particularly in protein expression after the tissue or cells have been removed from the subject. Changes in expression levels are known to change rapidly following perturbations, e.g., heat shock or activation with lipopolysaccharide (LPS) or other reagents. In addition, the RNA and proteins in the tissue and cells may quickly become degraded. Accordingly, tissues obtained from a subject are ideally immediately fixed or frozen. Tissue specimens may also include xenograft tumor samples, particularly those from animals in drug dose ranging or toxicology studies.

Quantitation. Any suitable method of quantifying or rating JMJD6 molecules may be used in the present methods. One preferred method utilizes immunohistochemistry, a staining method based on immunoenzymatic reactions using monoclonal or polyclonal antibodies to detect cells or specific proteins such as tissue antigens. Typically, immunohistochemistry protocols involve at least some of the following steps: 1) antigen retrieval (eg., by pressure cooking, protease treatment, microwaving, heating in appropriate buffers, etc.); 2) application of primary antibody and washing; 3) application of labeled secondary antibody that binds to primary antibody (often a second antibody conjugate that enables the detection in step 5) and wash; 4) an amplification step may be included; 5) application of detection reagent (e.g. chromagen, fluorescently tagged molecule or any molecule having an appropriate dynamic range to achieve the level of or sensitivity required for the assay); 6) counterstaining may be used and 7) detection using a detection system that makes the presence of the proteins visible (to either the human eye or an automated analysis system), for qualitative or quantitative analyses. Various immunoenzymatic staining methods are known in the art for detecting a protein of interest. For example, immunoenzymatic interactions can be visualized using different enzymes such as peroxidase, alkaline phosphatase, or different chromogens such as DAB, AEC, or Fast Red; or fluorescent labels such as FITC, Cy3, Cy5, Cy7, Alexafluors, etc. Counter stains may include H&E, DAPI, Hoechst, so long as such stains are compatible with other detection reagents and the visualization strategy used. As known in the art, amplification reagents may be used to intensify staining signal. For example, tyramide reagents may be used. The staining methods of the present invention may be accomplished using any suitable method or system as would be apparent to one of skill in the art, including automated, semi-automated or manual systems.

### Diagnosis

The expression of JMJD6 increases with increased metastasis of breast cancer. There is a 3 to 10 fold amplification of the amount of JMJD6 in an advanced and/or metastatic sample relative to the amount of JMJD6 in a control sample isolated from normal non-cancerous cells. Consequently, establishing the status of the amount of JMJD6 of an individual with possible breast cancer may be a useful diagnostic and/or prognostic tool.

Diagnostic and prognostic methods will generally be conducted using a biological sample obtained from a patient. A “sample” refers to a sample of tissue or fluid suspected of containing an analyte polypeptide from an individual including, but not limited to, e.g., plasma, serum, spinal fluid, lymph fluid, the external sections of the skin, respiratory, intestinal, and genitourinary tracts, tears, saliva, blood cells, organs, tissue including breast tissue and samples of in vitro cell culture constituents.

According to the diagnostic and prognostic methods of the present invention, alteration of the JMJD6 gene sequence expression taken from a cell or tissue suspected to be tumorigenic when compared to the JMJD6 gene sequence expression taken from a normal, non-cancerous cell or tissue may be detected using anyone of the methods described herein. In addition, the diagnostic and prognostic methods can be performed to detect the JMJD6 gene sequence expression and confirm the presence of a breast cancer or a predisposition to metastasis of breast cancer. An increase of the JMJD6 gene sequence expression is indicative of the presence of a breast cancer or a predisposition to metastasis of breast cancer.

Detection kits

Detection kits may contain antibodies, amplification systems, detection reagents (chromogen, fluorophore, etc), dilution buffers, washing solutions, mounting solutions, counter stains or any combination thereof. Kit components may be packaged for either manual or partially or wholly automated practice of the foregoing methods. In other embodiments involving kits, this invention contemplates a kit including compositions of the present invention, and optionally instructions for their use. Such kits may have a variety of uses, including, for example, imaging, stratifying patient populations, diagnosis, prognosis, guiding therapeutic treatment decisions, and other applications.

### Treatment Methods

Treatment” and “treat” and synonyms thereof refer to both therapeutic treatment and prophylactic or preventative measures, wherein the object is to prevent or slow down (lessen) a breast cancer metasatises.

As used herein a “therapeutically effective amount” of a compound will be an amount of active agent that is capable of preventing or at least slowing down (lessening) breast cancer metasatises. Dosages and administration of an antagonist of the invention in a pharmaceutical composition may be determined by one of ordinary skill in the art of clinical pharmacology or pharmacokinetics. See, for example, Mordenti and Rescigno, (1992) Pharmaceutical Research. 9:17-25; Morenti et al., (1991) Pharmaceutical Research. 8:1351-1359; and Mordenti and Chappell, “The use of interspecies scaling in toxicokinetics” in Toxicokinetics and New Drug Development, Yacobi et al. (eds) (Pergamon Press: NY, 1989), pp. 42-96. An effective amount of the antagonist to be employed therapeutically will depend, for example, upon the therapeutic objectives, the route of administration, and the condition of the mammal. Accordingly, it will be necessary for the therapist to titer the dosage and modify the route of administration as required to obtain the optimal therapeutic effect. A typical daily dosage might range from about 10 ng/kg to up to 100 mg/kg of the mammal's body weight or more per day, preferably about 1 μg/kg/day to 10 mg/kg/day.

The term “antagonist” is used in the broadest sense, and includes any molecule that partially or fully blocks, inhibits, or neutralizes a biological activity of JMJD6. Suitable antagonist molecules specifically include antagonist antibodies or antibody fragments, small interfering RNA of the invention, vaccines, and small organic molecules.

Antibodies, vaccines and siRNA produced according to the invention, as well as other molecules identified by the screening assays disclosed herein, can be administered for the treatment of Breast cancer in the form of pharmaceutical compositions. Thus, the present invention also relates to compositions including pharmaceutical compositions comprising a therapeutically effective amount of an antagonist to JMJD6. As used herein a compound will be therapeutically effective if it is able to affect JMJD6 expression or activity.

Pharmaceutical forms of the invention suitable for injectable use include sterile aqueous solutions (where water soluble) or dispersions and sterile powders for the extemporaneous preparation of sterile injectable solutions and or one or more carrier. Alternatively, injectable solutions may be delivered encapsulated in liposomes to assist their transport across cell membrane. Alternatively or in addition such preparations may contain constituents of self-assembling pore structures to facilitate transport across the cellular membrane. It must be stable under the conditions of manufacture and storage and must be preserved against the contaminating/destructive action of microorganisms such as, for example, bacteria and fungi.

The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as, for example, lecithin, by the maintenance of the required particle size in the case of dispersion and by the use of surfactants. Preventing the action of microorganisms in the compositions of the invention is achieved by adding antibacterial and/or antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized active ingredient into a sterile vehicle which contains the basic dispersion medium and the required other ingredients from those enumerated above. In the case of sterile powders for the preparation of sterile injectable solutions, the preferred methods of preparation are vacuum drying and freeze-drying, to yield a powder of the active ingredient plus any additional desired ingredient from previously sterile-filtered solution thereof.

When the active ingredients, in particular small molecules contemplated within the scope of the invention, are suitably protected they may be orally administered, for example, with an inert diluent or with an edible carrier, or it may be For oral therapeutic administration, the active compound may be incorporated with excipients and used in the form of buccal tablets, and the like. Such compositions and preparations should contain at least 1% by weight of active compound. The percentage of the compositions and preparations may, of course, be varied and may conveniently be between about 5 to about 80% of the weight of the unit. The amount of active compound in such therapeutically useful compositions is such that a suitable dosage will be obtained. Preferred compositions or preparations according to the present invention are prepared so that a dosage unit form contains between about 0.1 μg and 20 g of active compound. Of course, any material used in preparing any dosage unit form should be pharmaceutically pure and substantially non-toxic in the amounts employed. In addition, the active compound(s) may be incorporated into sustained-release preparations and formulations.

It is especially advantageous to formulate parenteral compositions in dosage unit form for ease of administration and uniformity of dosage. Dosage unit form as used herein refers to physically discrete units suited as unitary dosages for the mammalian subjects to be treated, each unit containing a predetermined quantity of active material calculated to produce the desired therapeutic effect in association with the required pharmaceutical carrier. The dosage unit forms of the invention are dictated by and directly dependent on (a) the unique characteristics of the active material and the particular therapeutic effect to be achieved, and (b) the limitations inherent in the art of compounding such an active material for the treatment of disease in living subjects having a diseased condition in which bodily health is impaired as herein disclosed in detail.

The principal active ingredient is compounded for convenient and effective administration in effective amounts with a suitable pharmaceutically acceptable carrier in dosage unit form. A unit dosage form can, for example, contain the principal active compound in amounts ranging from 0.5 μg to about 2000 mg. Expressed in proportions, the active compound is generally present in from about 0.5 pg to about 2000 mg/ml of carrier. In the case of compositions containing supplementary active ingredients, the dosages are determined by reference to the usual dose and manner of administration of the said ingredients.

### Candidate Substances

A candidate therapeutic substance may be a substance that modulates JMJD6 protein activity, and/or concentration preferably one that inhibits JMJD6 protein activity, and/or concentration. Candidate substances may conveniently be preliminarily screened by in vitro binding assays such as yeast to hybid assays as known in the art and then tested, for example in a whole cell assay as described below. Examples of candidate substances include antibodies which recognise JMJD6, or small interfering RNA that downreglates JMJD6 expression such as those defined in SEQ ID No. 9, SEQ ID No. 10 or SEQ ID No. 11 or a homologue variant, derivative or fragment polynucleotide thereof as defined above.

A substance which can bind directly to JMJD6 may also inhibit any interaction between JMJD6 and demethylation of an estrogen receptor gene. That is where it is shown there is a direct relationship between estrogen receptor and JMJD6 and histones. Non-functional homologues of JMJD6 may also be tested for inhibition of JMJD6 activity. Such non-functional homologues may include naturally occurring JMJD6 mutants and modified JMJD6 sequences or fragments thereof. In particular, fragments of JMJD6 which comprise one or more of a non-functional jumonji domain that can bind to methylated histones or other functional domains that may be used to compete with full length JMJD6.

Alternatively, instead of preventing the association of the components directly, the substance may alter the biologically available amount of JMJD6. This may be by inhibiting expression of the component, for example at the level of transcription, transcript stability, translation or post-translational stability. An example of such a substance would be antisense RNA or double-stranded interfering RNA sequences which suppresses the amount of JMJD6 mRNA biosynthesis such as those defined in SEQ ID No. 5, SEQ ID No.6 or SEQ ID No. 7. In particular, inhibition of JMJD6 protein may inhibit breast cancer tumourigenis or metastasis in vitro or in vivo.

Suitable candidate substances include peptides, especially of from about 5 to 30 or 10 to 25 amino acids in size, based on the sequence of the various domains of JMJD6 described above, or variants of such peptides in which one or more residues have been substituted. Peptides from panels of peptides comprising random sequences or sequences which have been varied consistently to provide a maximally diverse panel of peptides may be used.

Means of knocking out or knocking down JMJD6 protein may be used including siRNA an RNA interference sequence capable of interfering with JMJD6 gene expression; alternative RNA splicing techniques; posttranslational processing to JMJD6; the level of expression of JMJD6 protein including both mRNA expression and protein expression; or any mutation of JMJD6 protein that effects JMJD6 protein expression or translocation to the nucleus.

Suitable candidate substances also include antibody products (for example, monoclonal and polyclonal antibodies, single chain antibodies, chimeric antibodies and CDR-grafted antibodies) which are specific for JMJD6. Furthermore, combinatorial libraries, peptide and peptide mimetics, defined chemical entities, oligonucleotides, and natural product libraries may be screened for activity as regulators of JMJD6 expression. The candidate substances may be used in an initial screen in batches of, for example 10 substances per reaction and the substances of those batches which show inhibition tested individually. Candidate substances which show decreased JMJD6 expression in in vitro screens such as those described below can then be tested in whole cell systems, such as mammalian cells which will be exposed to the inhibitor and tested for effects on tumorigenesis.

### Examples of Preferred Embodiments

Samples were taken from six breast cancer cohorts. Each cohort included more than 100 subjects diagnosed with breast cancer. The expression patterns of JMJD6 in the samples were measured on an Affymetrix U133A genechip using the three Affimetrix probes sets that were identified as mapping to JMJD6: 212722_s_at and 212723_s_at, and 215233_at. The JMJD6 gene expression profile for one of the cohorts was measured on an Agilent microarray platform with the Agilent AB011157 probe. The expression patterns of JMJD6 were correlated by Cox proportional-hazards regression to the survival of patients among five independent breast cancer cohorts. JMJD6 was highly expressed and reproducibly correlated with poor survival in cohort subjects. A Cox regression/Kaplan-Meier analysis was used to assess the correlations between gene expression values of JMJD6 and the risk of distant metastasis. The correlation between increased JMJD6 gene expression and distant metastasis was statistically significant in two cohorts. The significance of the correlation between increased gene expression and distant metastasis is shown in table 1.

TABLE 1
significance of the correlation between increased gene expression and
distant metastasis in the breast cancer cohorts

| Name of | Number of | Significance Ratio | |
|---|---|---|---|
| Cohort | patients/cohort | test P-VALUE | Probe set ID |
| Uppsala | 251 | P = 0.07 | A.212722_S_at |
| Stockholm | 159 | P = 0.004 | A.215233_at |
| Oxford | 109 | P = 0.0008 | A.212723_at |
| Erasmus | 286 | P = 0.009 | A.212723_at |
| Mainz | 200 | P = 0.01 | A.212723_at |
| NKI | 295 | P = 0.004 | AB011157 |

The oncogenic potential of JMJD6 gene expression was tested in several commercially available breast cancer cell lines. In MCF-7, MDAMB231 and BT-549. The JMJD6 gene expression of the cells was knocked down using specific siRNA. siRNA 1—Sense GCUAUGGUGAACACCCUAATT; Antisense UUAGGGUGUUCACCAUAGCTG siRNA 2:—Sense GGUGCUUUCAGCGUAAGCUTT; Antisense AGCUUACGCUGAAAGCACCTC and siRNA 3:—Sense GGAUUAGGGACACUUGUGGTT; Antisense CCACAAGUGUCCCUAAUCCTC The Sequences are written as 5′->3′

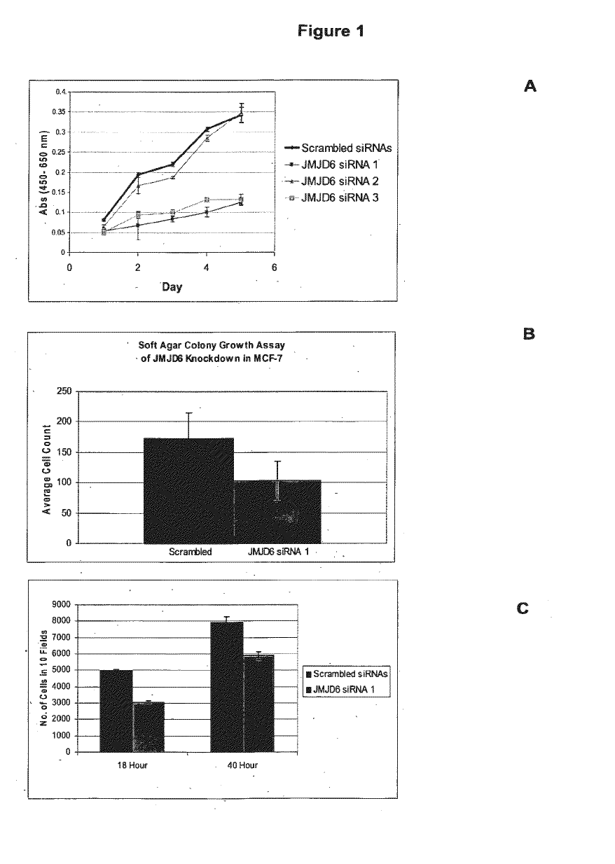

Cells with decreased expression for JMJD6 were assayed for four properties of cancer development. FIG. 1 demonstrates that all the oncogenic properties measured were decreased when JMJD6 was knocked down in cancer cell lines. Proliferation of cells was measured by Cell Proliferation Reagent WST-1, anchorage independent growth was measured by the cells ability to form colonies in soft agar, motility was measured by BD Matrigel™ Basement Membrane Matrix, Growth Factor Reduced (GFR), and invasion was measured by BD Falcon™ FluoroBlok™ Cell Culture Inserts for 24-well plates, 8.0 μm. The results suggest that JMJD6 is an oncogene in breast cancer.

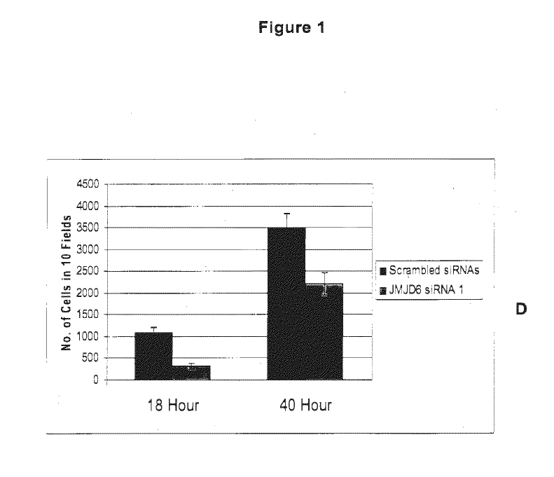

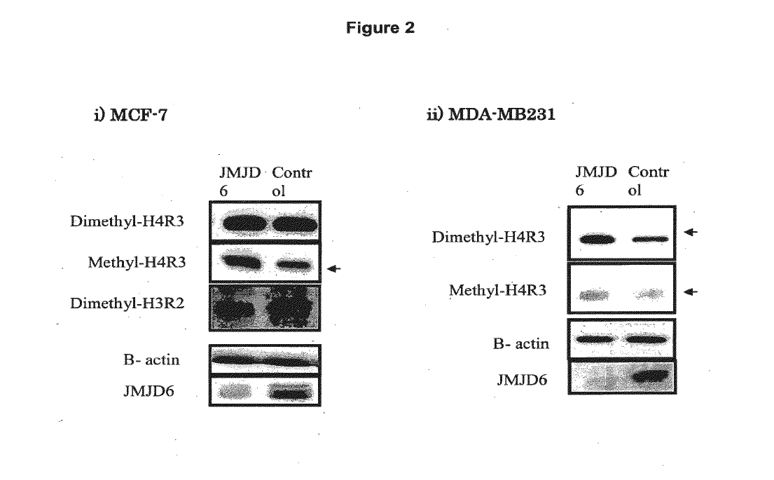

Without limiting ourselves to any particular theory we suggest that the oncogenecity of JMJD6 is closely related to the histone arginine demethylase activity of JMJD6. We depleted JMJD6 expression using siRNA strategies in MCF-7 and MDA-MB231 cells, and observed increased methylation of Histone H4R3 substrate but not H3R2 (FIG. 2). Moreover, increased JMJD6 was associated with an increase in estrogen receptor expression in MCF7 cells, whereas loss of JMJD6 led to the concurrent loss in estrogen receptor (ER) expression (FIG. 3). We have demonstrated that histone modifications at the lysine residues substantially influence ER binding site patterns in these cells. However, the importance of epigenetic changes associated with arginine residues remains poorly characterized. Previous reports suggest that histone arginine methylation patterns fine tune estrogen receptor-mediated transcriptional activity. As JMJD6 is a histone demethylase and may antagonize ER-mediated gene transcription, the compounds used could be exploited for advanced breast cancer therapy. The development of other small molecule inhibitors to JMJD6 could be used in the same way.

### In Vivo Effects of JMJD6

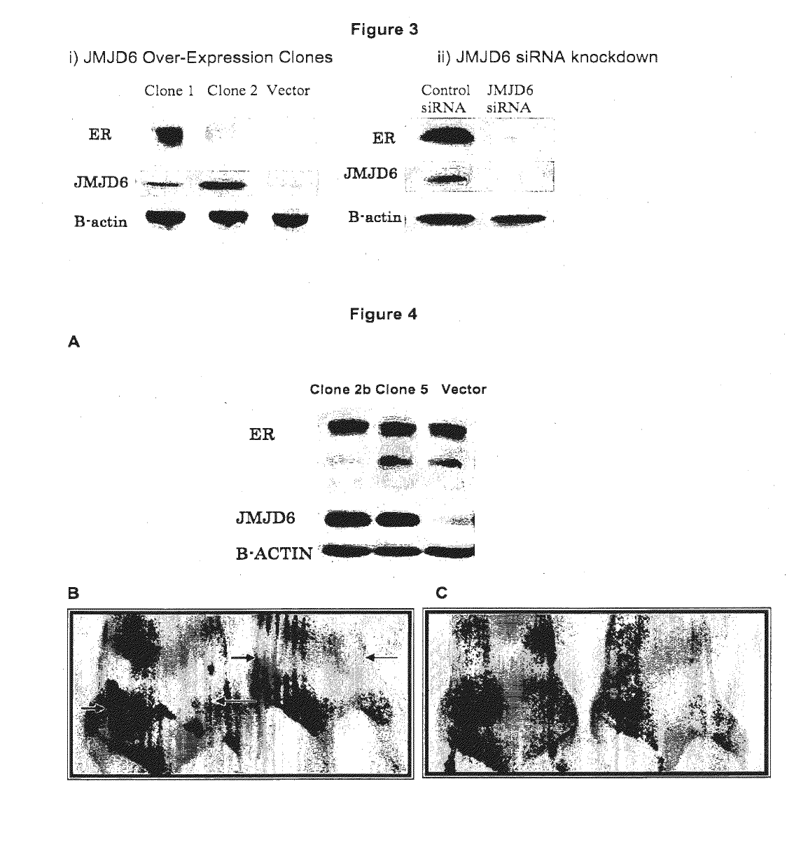

Our cell based assays suggest that JMJD6 is a potential oncogene. To test its ability to form solid tumors in vivo, we developed JMJD6 over expressing stable cell lines in MCF-7 cells (FIG. 4, panel A). 1×10⁶ cells (two clones and a control vector) were injected per flank in athymic nude mice. As athymic nude mice have sub-optimal hormone levels, MCF-7 cells need supplementary hormone (estradiol) pellets for growth in these mice. It is expected that an oncogene (JMJD6) transformed MCF-7 cells will grow larger tumors than the vector control. However, in contrast our data showed that adding of estrogen and the oncogene lead to cell death and no subsequent tumor formation in the JMJD6 expressing cells (panel B and C). These experiments suggest that change in JMJD6 levels leads to differential responsiveness of cells to estrogen treatment.

Association of gene expression signatures with poor survival identified JMJD6 as a strong candidate biomarker, whose high expression correlated with decreased metastasis free survival and it was validated as a candidate oncogene in cell-based assays. Secondly, its histone demethylase activity and influence on ER and ER mediated gene transcription has therapeutic value in treating early as well as advanced breast cancer. Knockdown of JMJD6 decreases demethylation of its substrates JMJD6 levels correlate with Estrogen receptor levels

Our research has demonstrated a causal relationship between estrogen receptor, JMJD6 expression levels and histone modification patterns. Together, the data presented indicates a complex pattern of Estrogen Receptor mediated response depending upon the increased or decreased levels of JMJD6 in the cells. Possibly histone modifications by JMJD6 substantially influence Estrogen Receptor binding site patterns in breast cancer cells. JMJD6 may be a histone arginine demethylase. However, the importance of epigenetic changes associated with arginine residues remains poorly characterized. Previous reports suggest that histone arginine methylation patterns fine tune estrogen receptor (ER)-mediated transcriptional activity at pS2/TFF1 promoter. CARM1 methyltransferase is recruited during transcriptional activation via estrogen receptor, while histone deimination by peptidyl arginine deiminase 4 (PADI4) antagonizes the arginine methylation. We propose to investigate if JMJD6, being the first and only arginine demethylase to be discovered to date, might parallel the effects of PADI4 in antagonizing estrogen-induced transcriptional activation. Towards this we have initiated chromatin immunoprecipitation experiments using JMJD6, histone arginine methylases (H4R3, H3R2) and ER to map the hormone binding and histone modification sites before and after estrogen treatment in breast cancer cells. ChIP material obtained will be used for high throughput sequencing. These experiments will allow the identification of JMJD6 transcriptional targets in cells and those that are co-regulated by ER. Another approach would be to perform a sequential ChIP with JMJD6 followed by ER antibodies and evaluation of ER targets that can be identified by real-time PCR analysis.

Successful initial ChIP experiments suggest that JMJD6 is localized onto or near the DNA and is associated with a few targets of ER. Secondly, physical interaction of JMJD6 and its target H4R3/H3R2 has been proposed. It is plausible that JMJD6 directly interacts with ER and/or other proteins of the transcriptional machinery either for recruitment to the DNA or enzymatic activity. To determine the complete molecular function of JMJD6, interacting partners of JMJD6 will be identified by mass spectrometric analysis of ChIP complexes and confirmatory co-immuno precipitation experiments.

In contrast to expected induction of tumor growth, cells over-expressing JMJD6 failed to form tumors in nude mice suggesting that they were somehow sensitized to estrogen treatment. These data suggest that there is a causal relationship between cell physiology, estrogen and perturbed levels of JMJD6.

We suggest that the Estrogen Receptor and JMJD6 both will be amplified in advanced metastatic breast cancer. However, clinically most metastatic cancers very little estrogen receptor expression. In PCR and immunohistochemistry as a ratio of high JMJD6 indicates advanced or metastatic cancer with or without amplification of estrogen receptor expression, and mostly samples with high JMJD6 and absence of ER will be metastatic. Amplification of both estrogen receptor expression and JMJD6 expression may still be metastatic. All traditional treatments including chemo/radiation therapy that are used where there is no amplification of estrogen receptor expression in breast tumors may be successfully used withJMJD6 antagonist in combination.

Those skilled in the art will appreciate that the invention described herein is susceptible to variations and modifications other than those specifically described. The invention includes all such variation and modifications. The invention also includes all of the steps, features, formulations and compounds referred to or indicated in the specification, individually or collectively and any and all combinations or any two or more of the steps or features.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness.

Any manufacturer's instructions, descriptions, product specifications, and product sheets for any products mentioned herein or in any document incorporated by reference herein, are hereby incorporated herein by reference, and may be employed in the practice of the invention.

The present invention is not to be limited in scope by any of the specific embodiments described herein. These embodiments are intended for the purpose of exemplification only. Functionally equivalent products, formulations and methods are clearly within the scope of the invention as described herein.

The invention described herein may include one or more range of values (eg size, concentration etc). A range of values will be understood to include all values within the range, including the values defining the range, and values adjacent to the range which lead to the same or substantially the same outcome as the values immediately adjacent to that value which defines the boundary to the range.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

While the invention has been described with reference to specific methods and embodiments, it will be appreciated that various modifications and changes may be made without departing from the invention.

1. A method of analyzing a cell expression profile for determining whether the cell is metastatic comprising the steps of:
a) Measuring an amount of Jumonji domain containing-6 nucleic (JMJD6) acid or polypeptide in a sample of the cell; and
b) Comparing the amount of JMJD6 nucleic acid or protein present in the cell to the amount of JMJD6 nucleic acid or polypeptide in a sample isolated from normal, non-cancerous cells,
wherein an amplified amount of JMJD6 nucleic acid or polypeptide in the cell relative to the amount of JMJD6 nucleic acid or polypeptide in the sample indicates metastatic breast cancer is present in the cell; and wherein the absence of an amplified amount of JMJD6 nucleic acid or polypeptide in the cell relative to the amount of JMJD6 nucleic acid or polypeptide in the sample indicates there is no metastatic breast cancer present in the cell.

2. A method of detecting a metastatic state of breast cancer comprising the steps of:
a) Measuring the amount of JMJD6 nucleic acid or polypeptide in a first biological sample; and
b) Comparing the amount of JMJD6 nucleic acid or polypeptide in the first sample with the amount of JMJD6 nucleic acid or polypeptide in a second biological sample isolated from normal, non-cancerous cells,
wherein an amplified amount of JMJD6 nucleic acid or polypeptide in the first biological sample relative to the amount of JMJD6 nucleic acid or polypeptide in the second biological sample indicates breast cancer has metastasized; and wherein the absence of an amplified amount of JMJD6 nucleic acid or polypeptide in the first biological sample relative to the amount of JMJD6 nucleic acid or polypeptide in the second biological sample indicates the breast cancer has not metastasized.

3. The method of claim 1 or 2 wherein the JMJD6 nucleic acid comprises nucleotide sequence SEQ ID NO 1 or SEQ ID NO 3 or SEQ ID NO 5.

4. The method of claim 1 or 2 wherein the JMJD6 polypeptide comprises sequence SEQ ID NO 2; or SEQ ID NO 4; or SEQ ID NO 6.

5. The method of any one of claim 1, 2 or 3 further comprising the steps of:
a) bringing the nucleic acid into contact with a polynucleotide probe or primer comprising a polynucleotide sequence capable of hybridising selectively to the nucleotide sequence set out in SEQ ID No. 1 or SEQ ID NO 3, or SEQ ID NO 5 or SEQ ID NO 7 under suitable hybridising conditions; and
b) detecting any duplex formed between the probe or primer and nucleic acid.

6. The method as claimed in claim 5 further comprising the step of detecting an estrogen receptor-encoding sequence wherein an amplified amount of JMJD6 nucleic acid and a variation in the amount of the estrogen receptor encoding sequence relative to the amount of JMJD6 nucleic acid and the estrogen receptor encoding sequence isolated from normal, non-cancerous cells indicates a breast cancer has metastasized; and wherein the absence of an amplified amount of the JMJD6 nucleic acid encoding sequence relative to the amount of JMJD6 nucleic acid and the estrogen receptor encoding sequence isolated from normal, non-cancerous cells indicates a breast cancer has not metastasized.

7. An antibody capable of binding selectively a JMJD6 polypeptide which comprises a sequence set out in SEQ ID No 2; or SEQ ID 4; or SEQ ID NO 6; or SEQ ID NO 8.

8. The method of any one of claim 1, 2, or 4 further comprising the steps of:
a) providing the antibody of claim 7;
b) incubating a biological sample with the antibody under conditions which allow for the formation of a first antibody-antigen complex; and
c) determining whether the antibody-antigen complex comprising the antibody is formed.

9. An immunhistochemical method for measuring a JMJD6 polypeptide in a test tissue section comprising the steps of:
a) incubating the test tissue section with the antibody of claim 7 under conditions which allow for the formation of a first antibody-antigen complex
b) staining the antibody-antigen complex of the test tissue section with a reagent; and
d) analyzing the image to quantify an amount of the stained antibody-antigen complex in the test tissue section;
wherein an amplified amount of the stained antibody-antigen complex relative to the amount of the stained antibody-antigen complex in a tissue section taken from normal, non-cancerous tissue indicates that the breast cancer has metastasized; and
wherein the absence of an amplified amount of the stained antibody-antigen complex relative to the amount of the stained antibody-antigen complex in a tissue section taken from normal, non-cancerous tissue indicates the breast cancer has not metastasized.

10. The method of claim 9 further comprising the steps of using an optical microscope, obtaining an image of the stained antibody-antigen complex in the test tissue section.

11. The method of claim 9 in which the test tissue section comprises a cell or plurality of cells suspected to be cancerous.

12. The method of any one of claims 9 to 11 in which the test tissue section is fixed.

13. The method of claim 8 or 9 wherein an additional second antibody capable of binding selectively a estrogen receptor polypeptide is incubated with the biological sample or the test tissue section under conditions which allow for the formation of a second antibody-antigen complex; and determining whether the second antibody-antigen complex is formed, wherein a loss of amount of the second antibody-antigen complex relative to the amount of the second antibody-antigen complex in a sample taken from normal, non-cancerous tissue in combination with an amplified amount of the first antibody-antigen complex relative to the amount of the first antibody-antigen complex in a sample taken from normal, non-cancerous tissue indicates a breast cancer has metastasized; and wherein the presence of an amplified amount of the second antibody-antigen complex relative to the amount of second antibody-antigen complex in the sample taken from normal, non-cancerous tissue in combination with an absence of an amplified amount of the first antibody-antigen complex relative to the amount of the first antibody-antigen complex in a sample taken from normal, non-cancerous tissue indicates a breast cancer has not metastasized.

14. A method of treating breast cancer comprising administering to a patient in need of therapy an antibody of claim 7.

15. The method of claim 14 further comprising administering an estrogen receptor antagonist.

16. The method of claim 15 wherein the estrogen receptor antagonist comprises tamoxifen.

17. The method of claim 15 wherein the estrogen receptor antagonist comprises raloxifene.

18. A composition comprising a therapeutically effective amount of an inhibitor of JMJD6 polynucleotide expression in cells

19. The composition of claim 18 wherein the inhibitor is the antibody of claim 7

20. The composition of claim 18 wherein the inhibitor is an interfering RNA.

21. The composition of claim 20 wherein the interfering RNA comprises sequences SEQ ID No. 9, or SEQ ID No. 10, or SEQ ID No. 11.

22. The composition of any one of claims 19 to 21 further comprising an estrogen receptor antagonist.

23. The composition of claim 22 wherein the estrogen receptor antagonist comprises tamoxifen.

24. The composition of claim 22 wherein the estrogen receptor antagonist comprises raloxifene.

25. The composition of any one of claims 18 to 24 for use in treating breast cancer.

26. A use of the composition of any one of claims 18 to 24 for the preparation of a medicament for the treatment of breast cancer.

27. A kit for detecting breast cancer in cells comprising a reagent for detecting JMJD6 polynucleotide expression; a buffer and instructions for detecting whether breast cancer cells have metastasized.

28. The kit of claim 27 wherein the reagent comprises the antibody of claim 7.

29. The kit of claim 28 further comprising an antibody capable of binding selectively to an estrogen receptor.

30. The kit of claim 28 wherein the reagent comprises a primer and a probe comprising a polynucleotide sequence capable of hybridising selectively to the nucleotide sequence set out in SEQ ID No. 1 or SEQ ID NO 3, or SEQ ID NO 5 or SEQ ID NO 7 under suitable hybridising conditions.

31. A method for screening for an antagonist of JMJD6 polynucleotide expression comprising the steps of:
a. contacting a cell expressing JMJD6 polynucleotide with a sample compound;
b. measuring the amount of JMJD6 polynucleotide expression in both the presence and absence of the sample compound; and
Wherein a decrease in JMJD6 polynucleotide expression in the presence of the sample compound in relation to the JMJD6 polynucleotide expression in the absence of the sample compound indicates the sample compound is the antagonist.

32. A method for screening for an antagonist of JMJD6 polypeptide activity comprising the steps of:
a. contacting a cell expressing JMJD6 polynucleotide with a sample compound;
b. measuring the activity of JMJD6 polynucleotide expression in both the presence and absence of the sample compound; and
Wherein a decrease in JMJD6 polypeptide activity in the presence of the sample compound in relation to the JMJD6 polynucleotide expression in the absence of the sample compound indicates the sample compound is the antagonist.

33. A method of making an antibody specific for JMJD6 polypeptide comprising the steps of:
(a) isolating a JMJD6 polypeptide from a metastatic breast cancer;
(b) conjugating a JMJD6 polypeptide to a carrier protein;
(c) inducing production of an antibody of the JMJD6 polypeptide—carrier protein conjugate in a cell; and
(d) obtaining the antibody from the cell

34. The method of claim 33 wherein the cell comprises a host animal induced by immunisation.

35. The method of claim 34 further comprising adding an adjuvant with the JMJD6 polypeptide—carrier protein conjugate prior to immunizing the host animal.

36. The method of claim 33 wherein the cell comprises a hybridoma.

37. A vaccine for treating metastatic breast cancer comprising a JMJD6 polypeptide.

38. The vaccine of claim 37 further comprising at least one suitable adjuvant.

39. The vaccine of claim 37 or 38 wherein the JMJD6 polypeptide comprises a sequence set out in SEQ ID No 2; or SEQ ID 4; or SEQ ID NO 6.

40. A method of treating breast cancer comprising administering to a patient in need of therapy a vaccine of and one of claims 37 to 39.

41. The vaccine of any one of claims 37 to 39 for use in treating breast cancer.

42. A use of the vaccine of any one of claims 37 to 39 for the preparation of a medicament for the treatment of breast cancer.
